(19) United States
(12) Patent Application Publication
(10) Pub. No.: US 2024/0285742 A1
(43) Pub. Date: Aug. 29, 2024

(54) Universal mRNA Antimalarial Vaccine

(71) Applicants: Sarfaraz K. Niazi, Deerfield, IL (US); Matthias Magoola, Kampala (UG); Zafeer Ahmad, Wadsworth, IL (US)

(72) Inventors: Sarfaraz K. Niazi, Deerfield, IL (US); Matthias Magoola, Kampala (UG); Zafeer Ahmad, Wadsworth, IL (US)

(73) Assignee: RNA Therapeutics, Inc., Deerfield, IL (US)

(21) Appl. No.: 18/398,119

(22) Filed: Dec. 27, 2023

(51) Int. Cl. A61K 39/015; A61K 39/00

(52) U.S. Cl. CPC A61K 39/015; A61K 2039/53; A61K 2039/55555; A61K 2039/572; A61K 2039/575; C12N 2310/532

(57) ABSTRACT

An mRNA composition for malaria prevention and treatment comprising a coding mRNA capable of expressing proteins and peptides of malaria pathogens, coming from linear epitopes and protein structure of antigens.

### SEQUENCE LISTING

The instant application contains a Sequence Listing, which has been submitted electronically in ASCII format and is hereby incorporated by reference in its entirety. Said ASCII text file, created on May 19, 2024, is named Universal Vaccine N, and is 19.3 kb in size.

### INTRODUCTION

Malaria is an infectious disease caused by Plasmodium spp—parasites, transmitted by bites from infected female Anopheles mosquitoes. According to the World Health Organization (WHO), globally, there were an estimated 247 million malaria cases in 2021 in 84 malaria-endemic countries (including the territory of French Guiana), an increase from 245 million in 2020, with most of this increase coming from countries in the WHO African Region. To control the disease more effectively, different strategies have been employed to control the disease, but the ineffectiveness in controlling vectors and parasite resistance to antimalarial drugs suggest that the development of a vaccine as a preventive measure could be important. Currently, the main pillars for the management of malaria are rapid diagnostic tests and artemisinin derivatives for treatment. However, these strategies have not been enough to maintain a downward trend in malaria incidence and mortality. Recent findings in the pathophysiology of malaria have highlighted the importance of the host's response to the infection.

Many malaria parasites are now immune to the most common drugs used to treat the disease. According to the malaria Eradication Research Agenda initiative, malaria eradication will be only achievable through effective vaccination.

An effective malaria vaccine should induce strong humoral immune responses and CD8+ T-cell responses. Therefore, an effective malaria vaccine should ideally provide an antigen in the N-terminal region for strong induction of a CD8+ T-cell response and the B-cell response.

An epitope, also known as an antigenic determinant, is a specific part of an antigen recognized and bound by an antibody, B-cell receptor, or T-cell receptor during an immune response. Antigens are substances (often proteins) that can trigger an immune response in the body, and they may be part of a pathogen such as a virus or bacterium or a foreign substance like pollen. Epitopes can be categorized into two main types. The linear or sequential epitopes consist of a linear sequence of amino acids within the antigen's primary structure. They are recognized by their amino acid sequence rather than their three-dimensional structure. The conformational or discontinuous epitopes are formed by amino acids that are not sequential in the primary protein sequence but are brought together in space by the protein folding. These epitopes are recognized by their three-dimensional structure. Given any amino acid sequence, the B and T-cell epitopes can be calculated (http://tools.iedb.org).

The interaction between an epitope and an antibody or a receptor is specific; a particular epitope will bind to a specific antibody or receptor with high specificity. This specificity is a fundamental principle of the immune system's ability to detect and respond to various antigens. Understanding epitopes is crucial in various fields, including vaccine development, immunotherapy, and diagnostic testing.

Epitopes are the specific regions of antigens (in this case, the autoantibodies) that are recognized by autoantigens. For MHC binding, epitopes must bind to major histocompatibility complex (MHC) molecules to be presented to T cells. For class I MHC, epitopes are typically 8-11 amino acids in length, while for class II MHC, they are usually longer, around 15-24 amino acids. Some epitopes may be discontinuous and composed of amino acids not adjacent to the protein sequence. B cell epitopes are usually 5-17 amino acids in length but can be extended without adverse effects on the immune function.

B cell epitopes are typically fragments located on the outer surface of a (native) protein or peptide antigens, preferably having 8 to 15 amino acids, which may be recognized by antibodies, i.e., in their native form. This invention used a 5-25 amino acid sequence cutoff since epitopes are rarely found beyond these limits. Such epitopes of proteins or peptides may be selected from any of the variants of such proteins or peptides. In this context, antigenic determinants can be conformational or discontinuous epitopes, which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain. In the context of the present invention, an epitope may be the product of the translation of a provided coding mRNA as specified herein.

### BACKGROUND OF THE INVENTION

Advantageously, mRNA can be manufactured in a large-scale fashion and enables the production of a robust immune response based on mRNA encoding, for example, antigens that produce antibodies specific to proteins of the target infecting cell.

In various embodiments, the coding mRNA comprises, preferably in 5′- to 3′-direction, the following elements:
5′-cap structure selected from m7G(5′), m7G(5′)ppp(5′)(2OMeA), or m7G(5′)ppp(5′)(2′OMeG);
   A) The 5′-terminal start element was selected from AUG, GUG, and UUG.
   B) 3-UTR
   C) poly(A) sequence comprising about 50 to about 500 adenosines.

### Cap

The 5′ end of the mRNA contains a 7-methylguanosine (m7G) moiety, followed by a triphosphate moiety to the first nucleotide (m7GpppN). m7GpppN is called a 5′ cap, a protective structure that protects RNA from exonuclease cleavage, regulates pre-mRNA splicing, and initiates mRNA translation and nuclear export of the mRNA to the cytoplasm. The 5′ cap is also essential in recognizing non-self mRNA or exogenous mRNA from self mRNA or the endogenous mRNA by the innate immune system.

The mRNA can be modified to improve its efficacy and stability by introducing many post-transcriptional modifications. Some of these include 2′-O-methylation at position 2′ of the ribose ring at the first nucleotide (Cap 1, m7GpppN1m) and the second nucleotide e (Cap 2, m7GpppN1mN2m) as well. These modifications in the 5′ cap structure not only increase the translation efficiency of mRNA but also stop the activation of endosomal and cytosolic receptors, including RIG-I and MDA5, which act as defensive mechanisms against viral mRNA.

Hence, the 2′-O-methylation of the 5′ cap structure is a highly desirable property for increasing and enhancing the protein production from the mRNA after its transcription and blocking any undesirable immune responses from the host immune system to the antigenic IVT mRNA. This 5′ cap can be achieved by adding S-adenosyl methionine and the Cap 0 structure to the IVT mRNA reaction, which yields IVT mRNA with the Cap 1 structure and S-adenosyl-L-homocysteine. Cap 1 refers to m7GpppNm, where Nm represents any nucleotide with a 2′O methylation. This structure plays a crucial role in RNA stability and the initiation of protein synthesis. Let's break down its components:
a) m7G: This represents a 7-methylguanosine residue. It's a modified guanine nucleotide with a methyl group attached to the nitrogen at the 7th position. This modification is crucial for RNA stability and efficient translation.
b) ppp: This stands for a triphosphate bridge. It connects the 5′ end of the mRNA with the m7G cap. This linkage is unusual because it's a 5′-to-5′ triphosphate linkage, unlike the typical 5′-to-3′ phosphodiester bonds in RNA.
c) Am: This signifies a 2′-O-methyladenosine residue. It's a modification where a methyl group is added to the 2′ hydroxyl group of the first nucleotide of the mRNA adjacent to the cap. This modification can enhance the stability of the mRNA and also plays a role in distinguishing self-RNA (e.g., from a cell's genes) from non-self-RNA (e.g., viruses or other pathogens) in the immune response.

The cap1 structure (m7GpppAm) is a common feature in eukaryotic mRNA and is essential for various aspects of RNA metabolism, including RNA stability, export from the nucleus, and translation initiation. It also helps in the recognition of the mRNA by the ribosome and other components of the translation machinery. In the modified structure, there's an additional methyl group at the 3′ position of the m7G cap (m7G+m3′). This modification might further influence the interaction of the cap with cellular proteins and potentially affect mRNA stability and translation efficiency. Such modifications are often explored in the context of mRNA-based therapeutics and vaccines, where they can enhance the stability and translational efficiency of the

An example of the modified 5′-cap1 structure (m7G+m3′-5′-ppp-5′-Am) can be found in certain messenger RNAs (mRNAs) used in mRNA-based vaccines, such as those developed for COVID-19. In these vaccines, the mRNA carries the instructions to produce a specific viral protein (like the spike protein of the SARS-CoV-2 virus) that triggers an immune response in the body. The modified cap structure plays a crucial role in these mRNA molecules.

The various types of CAPs that can be beneficial in the present invention include: ARCA, Bridged Cap (BCAP), Cap0, Cap1, Cap2, Cap3, Cap4, CleanCap, Hypermodified Caps, Modified Cap1, Synthetic or Designer Caps, Tobacco Mosaic Virus (TMV) Cap, and Viral Caps

### DETAILS OF THE INVENTION

The nucleoside sequence can be obtained by first converting the target protein into DNA through reverse transcription and then to RNA. Multiple epitopes can be linked together using linkers including but not limited to: AAAGY (Alanine-Alanine-Alanine-Glycine-Tyrosine), AAY (alanine and tyrosine), APAAP (Alanine-Proline-Alanine-Alanine-Proline), EAAAK (Glutamic Acid-Alanine Linker), EFGGG (Glutamic Acid-Phenylalanine-Glycine-Glycine-Glycine), GGAGG (A slight variation of the GGGGS linker with an alanine residue), GGGGS (Glycine-Serine Linker), GGGS (linker is one of the simplest and commonly used linkers), GGGSGGG (linker consists of a longer sequence of glycine (G) and serine (S) residues), GGGSGGGGSGGG (linker with multiple glycine and serine residues), GGPGG (Glycine-Glycine-Proline-Glycine-Glycine), GGSGG: (An inversion of the standard GGGGS linker), GGSSG (Glycine-Glycine-Serine-Serine-Glycine), GGTGG (Glycine-Glycine-Threonine-Glycine-Glycine), GPGP (Glycine-Proline-Glycine-Proline), GPGPG (Glycine-Proline-Glycine-Proline-Glycine), GPGS (Glycine-Proline-Glycine-Serine), GSGPG (Glycine-Serine-Glycine-Proline-Glycine), GSSG (Glycine-Serine-Serine-Glycine), GSSGG (Glycine-Serine-Serine-Glycine-Glycine), GSTSG (Glycine-Serine-Threonine Linker), KK (Lysine-Lysine), KKKGS (Lysine-Glycine-Serine Linker), KLPGWSG: (A specific sequence), LEGGGS (Leucine-Glutamic Acid-Glycine-Glycine-Serine), NPGP (Asparagine-Proline-Glycine-Proline), SGGGG: (A variant of the GGGGS linker), SGSGS (Serine-Glycine-Serine-Glycine-Serine), SSGGG (Serine-Serine-Glycine-Glycine-Glycine), SSGSS (Serine-Serine-Glycine-Serine-Serine), TGGGS (Threonine-Glycine-Glycine-Glycine-Serine), TPGTG (Threonine-Proline-Glycine-Threonine-Glycine), TPP (Proline-Proline-Threonine), TPTPPT (Threonine-Proline-Threonine-Proline-Proline-Threonine), TSGSG: (A variant of the GSTSG linker), TSGTSG (Threonine-Serine-Glycine-Threonine-Serine-Glycine), and XTEN: (A synthetic, non-immunogenic linker).

### UTRs

Although UTRs are not translated into the desired antigen or a protein, they regulate mRNA expression. These regions are located between the ORF and the 5′ and 3′ ends, in the upstream and the downstream of the mRNA. These UTRs contain regulatory sequences associated with mRNA stability and efficient and correct mRNA translation. They also help recognize mRNA by ribosomes and help in post-transcriptional modification of the mRNA. The mRNA translation and its half-life can be improved by including cis-regulatory sequences in the UTRs. Additionally, the inclusion of naturally occurring sequences, such as those derived from alpha- and beta-globins, have been widely used to design mRNA constructs for vaccines.

### Poly (A) Tail

The IVT mRNA has a polyadenylated section at its 3′ end known as the poly(A) tail. This polyadenylated tail is essential for determining the lifespan of the mRNA. The poly(A) tails of the naturally occurring mRNA molecules in mammalian cells have a longer length of approximately 250 nucleotides (nt), gradually shortened throughout the lifespan of mRNA in the cytosol. Since the tail size affects the degradation of mRNA, incorporating poly(A) tails is desirable in producing mRNA vaccines and therapeutics with a longer half-life. The addition of approximately 100 nt to the poly(A) tail can produce mRNA with the desired prolongation of degradation.

mRNA Delivery

mRNA vaccine molecules are large (104-106 Da) and negatively charged. They are unable to pass through the lipid bilayer of cell membranes. Naked mRNA would be destroyed and degraded by the nucleases in the bloodstream. In addition, naked mRNA is also attached and engulfed by immune cells in the tissue and the serum. Methods to deliver mRNA molecules into the cells include gene guns, electroporation, and ex vivo transfection. The in vivo methods of delivering mRNA involve transfection immune or non-immune cells using lipids or transfecting agents.

Although naked mRNA, liposomes, and polyplexes have shown clinical effectiveness in humans, LNPs for mRNA vaccines are the only drug delivery system that has demonstrated clinical efficacy and has been approved for human use. The COVID-19 mRNA vaccines against SARS-CoV-2, developed by Moderna and Pfizer/BioNTech, employ LNPs to deliver the mRNA payload to the body. LNPs are currently the foremost non-viral delivery vector employed for gene therapy. The clinical effectiveness of LNPs was first demonstrated when LNP-siRNA therapeutic Onpattro® (patisiran) was approved by the US FDA for hereditary transthyretin-mediated amyloidosis [34]. LNP formulations are the most successful, effective, and safe method of delivery of mRNA vaccines for human immunizations. LNPs offer numerous advantages for mRNA delivery to the site of action, including ease of formulation and scale-up, highly efficient transfection capacity, low toxicity profile, modularity, compactivity with different nucleic acid types and sizes, protection of mRNA from internal degradation, and increasing the half-life of mRNA vaccines. LNPs are typically composed of four components: an ionizable cationic lipid, a helper phospholipid, cholesterol, and a PEGylated lipid. These lipids encapsulate the mRNA vaccine's payload and protect the nucleic acid core from degradation.

### Cationic and Ionizable Lipids

Cationic lipids were the first generation developed and utilized for mRNA vaccine delivery. These lipids contain a quaternary nitrogen atom, imparting them a permanently positive charge. The positive charge of these lipids enables them to form ionic interactions with the negatively charged mRNA vaccines, forming a lipid complex called a lipoplex. DOTMA and its synthetic analog DOTAP were the first cationic lipids to deliver mRNA vaccines. Cationic lipids such as DOTMA, DOPE, and DOGS have been widely used for mRNA delivery since then, including the commercially available and successful Lipofectin, a mixture of DOPE and DOTMA, and is one of the first LNP formulations, proving successful in vivo translation of mRNA.

The early cationic lipids demonstrated promising gene delivery in vitro but suffered from inadequate in vivo efficacy. The positive charge of the nitrogen head group and the non-biodegradable nature of the early cationic lipids were responsible for their ineffective delivery and efficacy in vitro [40]. Ionizable lipids, also called pH-dependent ionic lipids, are the second generation of cationic lipids containing a primary amine, which imparts them a positive charge at or below physiological pH. The property of these lipids, having a neutral charge in the bloodstream at physiological pH, helps improve their safety compared to first-generation cationic lipids. They also extend the circulation time of the LNPs as compared to LNPs derived from cationic lipids. These were developed to overcome the shortcomings and safety issues, such as immune activation and interaction with serum proteins of the first-generation cationic lipids. DLin-MC3-DMA was the first US FDA-approved ionic lipid used in the first siRNA drug, Onpattro®. The DLin-MC3-DMA ionic lipid was synthesized after a series of modifications on the first ionic lipid, DODMA. DLinDMA was formed by replacing the oleyl tails of DODMA [42,43]. DLinDMA demonstrated superior ability to DODMA in protective immunity against the respiratory syncytial virus (RSV) in vivo. DLinDMA is further optimized to DLin-KC2-DMA and DLin-MC3-DMA depending on a series of structure-activity relationship-based studies. DLin-MC3-DMA is considered the first generation of ionizable lipids.

Including ester moieties helped increase the biodegradability of MC3 and increased its systemic clearance. Ester moieties are easy to install in a lipid, biodegradable, chemically stable, and easily cleaved by the intracellular esterase. MC3 was an essential precursor and a starting point for developing biodegradable ester ionizable lipids. Ester-based biodegradable ionizable lipids have demonstrated higher potency in gene delivery than MC3.

The third-generation ionizable lipids are optimized, having a limited number of chemical synthesis steps, which increases the high-throughput production of the ionizable lipids; 98N12-5 is the first example of a third-generation ionizable lipid.

### PEG-Lipid

Among the ingredients, polyethylene glycol (PEG) is a hydrophilic material well known for various cosmetic, food, and pharmaceutical applications. The PEGylated lipid component in LNPs is usually linked to an anchoring lipid. PEG was found to be an essential chemical in the formulation of LNPs to mitigate the uptake of nanoparticles by filter organs, also improving the colloidal stability of LNPs in biological fluids. Hence, circulation half-life and in vivo distribution of LNPs are enhanced.

Usually, PEG-lipids account for a minimal molar % among lipid constituents in LNPs (approximately 1.5%). However, they are pivotal in affecting crucial parameters such as population size, polydispersity index, aggregation reduction, particle stability improvement, and encapsulation efficiency. The molecular weight of PEG and the carbon chain length of the anchor lipid can be exploited to fine-tune the time of circulation and uptake by immune cells, altering the efficiency.

Additionally, the PEG-lipid coat on LNPs acts as a steric hydrophilic barrier for preventing self-assembly and aggregation during storage. Therefore, the presence of PEG helps stabilize the LNP and regulates size by limiting lipid fusion. The amount of PEG is inversely proportional to the size of the LNP; the higher the PEG content, the smaller the size of the LNP. Generally, the molecular weight of PEG ranges between 350 and 3000 Da, and the carbon chain of the anchored lipid lies between 13 and 18 carbon. Multiple literature reports indicated that a higher molecular weight of PEG and a longer lipid chain increase nanoparticle circulation time and reduce immune cell uptake.

As the PEG-lipid dissociates from the LNP surface, it decreases the circulation time of the LNP. It provides more chances for delivering the mRNA cargo into target cells by an effect called “PEG-Dilemma.” In some instances, the molar % of the PEG-lipid is maintained at 1.5%. The in vivo transfection level was found to be independent of the carbon chain length of the lipid. An added advantage of PEG-lipids relies on their capability of conjugating a specific ligand to the LNP, thus aiding in targeted drug delivery.

### Helper Lipids

The primary function of helper lipids in the formulation of LNPs lies in supporting their stability during storage and in vivo circulation. Chemically, these are glycerolipids and non-cationic. Among the various helper lipids, sterols and phospholipids are the most widely used. Cholesterol is a natural component present in cell membranes. It is an exchangeable moiety that can be easily accumulated in the LNP. Different studies have indicated that cholesterol might be present on the surface, within the lipid bilayer, or even conjugated with the ionized lipid within its core. It is usually incorporated in LNP formulation to maintain stability by filling gaps between lipids. Cholesterol is needed to regulate the density, uptake, and fluidity of the lipid bilayer matrix within the LNP.

Therefore, it controls the rigidity and integrity of the membrane, thereby preventing any leaks by the “condensing effect.” The hydrophobic tail, sterol ring flexibility, and polarity of hydroxy groups in cholesterol were reported to impact the efficacy of LNP delivery. Cholesterol also improves the circulation half-life of LNPs by reducing the surface-bound protein. Moreover, it helps by fusing with the endosomal membrane during the cellular uptake of LNPs. It plays a vital role in lowering the temperature needed for transitioning from the lamellar phase to the hexagonal phase; therefore, the mRNA cargo from the LNP will be delivered to the cytosol.

Including phospholipids in LNP formulation can help boost encapsulation (together with cholesterol) and increase cellular delivery. The number of phospholipids in the LNP is generally considerably reduced, increasing the cholesterol content for longer circulation times. Additionally, including phospholipids promotes the entrapment efficiency and transfection potency of the LNP. It has been reported that increasing the molar percentage of phospholipids contributes to expediting the efficacy of delivery by LNPs. These phospholipids in Zwitter ionic form have been reported to play a pivotal role in the assembly of the LNP through the stabilization of electrostatic interactions between the cationic lipid, mRNA cargo, and surrounding water molecules. However, the actual role of phospholipids in the delivery of mRNA via LNPs is still ambiguous.

mRNA Vaccines Manufacturing

mRNA vaccine production can be divided into three phases: upstream mRNA manufacturing, downstream mRNA purification, and formulation of mRNA lipid nanoparticles. mRNA production can be performed in a one-step co-transcriptional reaction, where a capping reagent is used, or in a two-step reaction, where the enzymatic capping is performed. mRNA purification process at a smaller lab-scale consists of DNase I digestion enzyme followed by LiCl precipitation of the mRNA. Purifying mRNA at a large scale involves utilizing well-established chromatographic methods coupled with tangential flow filtration (TFF). Finally, the formulation of mRNA vaccines consists of mixing an aqueous mRNA solution with a lipid solution in a non-aqueous phase. This causes the self-assembly of the lipid nanoparticles (LNPs) and encapsulates the negatively charged mRNA within the core of the LNPs. Mixing the mRNA and the lipid molecules in a staggered herringbone micromixer (SHM) occurs in various cycles, forming the final mRNA-LNP vaccines.

### Upstream Production

The upstream production of mRNA vaccines comprises the generation of the mRNA transcript from the plasmid containing the gene of interest. This reaction is called the in vitro transcription reaction (IVT). The IVT enzymatic reaction relies on RNA polymerase enzymes such as T7, SP6, or T3. The RNA polymerase enzymes catalyze the synthesis of the target mRNA from the linearized DNA template containing the gene of interest. A linearized DNA template is produced by the cleavage of a plasmid containing the gene of interest by restriction of endonuclease enzymes, or amplification of the gene of interest by PCR can also produce mRNA molecules.

The essential enzymes of an IVT reaction include (i) RNA polymerase-which converts DNA to RNA; (ii) inorganic pyrophosphatase (IPP)-increases IVT reaction yield, (iii) guanylyl transferase—adds GMP nucleoside to 5′ ends of mRNA, (iv) Cap 2′-O-Methyltransferase (SAM)—this enzyme adds a methyl group at the 2′ position of the 5′ cap of the mRNA, (v) DNase I—endonuclease used for removal of contaminating genomic DNA from RNA samples and degradation of DNA templates in the IVT reaction, and (vi) poly(A) tail polymerase and (vii) modified and unmodified nucleoside triphosphates (NTPs). These enzymes facilitate the upstream development of the mRNA transcript from a plasmid containing the gene of interest.

The capping enzymes include SAM and guanylyl transferase, which enzymatically form a 5′ cap at the 5′ end of the mRNA. In contrast, the poly(A) tail polymerase tailing enzyme forms the poly(A) tail. Another method of 5′ capping uses the co-transcriptional method, where the 5′ cap is prepared previously, and this cap is added to the mRNA non-enzymatic. This co-transcription reaction can be performed using CleanCap® Reagent AG.

### Downstream Purification

mRNA is produced by the IVT reaction in the upstream production phase; it is then isolated and purified by multiple purification steps in downstream processing. The IVT reaction mixture contains several impurities, including residual NTPs, enzymes, incorrectly formed mRNAs, and DNA plasmid templates. Lab-scale purification of IVT mRNA involves methods based on DNA removal by DNase enzyme digestion followed by lithium chloride (LiCl) precipitation.

The lab-based methods do not allow the complete removal of aberrant mRNA species, including dsRNA and truncated RNA fragments. Removing these impurities is essential and critical to obtaining a pure mRNA product that demonstrates its intended efficacy and safety profile. Yields 10-1000-fold can increase mRNA transfection and related protein production if reverse-phase HPLC purifies modified mRNA before its delivery to dendritic cells.

Chromatography is a commonly and widely used purification process accepted in the biopharmaceutical industry for purifying vaccines and biological drug products. The first published procedure in 2004 for large-scale nucleic acid purification of RNA oligonucleotides used size exclusion chromatography (SEC). SEC has several advantages, including selectivity, scalability, versatility, cost-effectiveness, and high purity and yields for nucleic acid products.

However, SEC cannot remove the same size impurities, such as dsDNA. Instead of SEC, ion-pair reverse-phase chromatography (IEC) is an excellent purification technique for mRNA vaccines. IEC can easily separate the target mRNA from the IVT reaction impurities. This separation method relies on the charge difference between the target mRNA and the impurities.

IEC has several advantages, including separating longer RNA transcripts from the target mRNA, higher binding capacity, cost-effectiveness, and scalability. The process becomes complex and temperature-sensitive since IEC is performed under denaturing conditions [84]. Affinity-based chromatographic separation is another mRNA purification method. Deoxythymidine (dT)-Oligo dT is a sequence that captures the mRNA's poly(A) tail. Chromatographic beads containing Oligo dT can be used for the downstream purification of mRNA vaccines.

Tangential flow filtration (TFF) or core bead filtration can remove small-sized impurities. As a final polishing step for mRNA vaccines, hydrophobic interaction chromatography (HIC) connected to a connective interaction media monolith (CIM) column containing OH or S03 ligands can be extremely beneficial.

### Formulation

mRNA molecules, being negatively charged, should be formulated in a lipid-based drug delivery system to avoid mRNA degradation and improve its transfection efficiency and half-life. LNPs are the most trustworthy, reliable, and US FDA-approved lipid-based non-viral carrier system for delivering mRNA vaccine drug substances. mRNA LNPs are formed by precipitating lipids dissolved in an organic phase and mixing them with mRNA in an aqueous phase. The most used lipids in the organic phase are ionizable, cholesterol, helper lipids, and PEG-lipids.

Meanwhile, the mRNA is dissolved in a citrate or acetate buffer at pH 4. Mixing the aqueous and non-aqueous solutions protonates the ionizable lipid, causing an electrostatic attraction between the ionizable protonated lipid and the anionic mRNA. This interaction is simultaneously coupled with the hydrophobic interactions of other lipids. It drives spontaneous self-assembly of the mRNA-LNPs with the mRNA encapsulated within the core of the nanoparticles. This process is also called microprecipitation. Following LNP formation, they are dialyzed to remove the non-aqueous solvent, usually ethanol, and the solution pH is elevated to physiological pH.

Microfluidic mixers enable the formation of small-sized LNPs with a low polydispersity index and high mRNA encapsulation efficiency. Microfluidic mixing is the most used method for mRNA LNP formulation at the lab scale and for GMP level. The Precision NanoSystems' NanoAssemblr® platform has been widely used for LNP formulation development and GMP production under controlled environments. This system uses a staggered herringbone micromixer (SHM) cartridge architecture. The structure of SHMs enables the two aqueous and non-aqueous solvents to mix within microseconds. This timescale is much smaller than the time required for lipid aggregation; hence, SHMs produce small nanoparticles of uniform size.

The NanoAssemblr® settings can be simply adjusted to change the aqueous and non-aqueous phase's flow rate and volume to obtain LNPs of the desired size and size distribution. A total flow rate of 12-14 mL/min and a flow rate volume ratio of 3:1, non-aqueous: aqueous phase, is commonly used to generate small monodisperse LNPs. Although SHMs have several advantages for efficient production of LNPs, their utility in GMP manufacturing is limited due to solvent incompatibility. The long-term exposure of the SMH and its internal parts containing polydimethylsiloxane to ethanol can lead to its deformation. It becomes difficult to replace the cartridges in a continuous GMP manufacturing run. Hence, T-mixers are utilized for LNP scale-up and manufacturing. They can produce LNPs like the SMH, handle higher flow rates and volumes (60-80 mL/min), and are compatible with organic solvents such as ethanol.

### PRIOR ART

There are countless challenges to the development of such a vaccine directly related to the parasite's complex life cycle (Table 1). After more than four decades of basic research and clinical trials, the World Health Organization (WHO) has recommended the pre-erythrocytic Plasmodium falciparum (RTS, S) malaria vaccine for widespread use among children living in malaria-endemic areas.

Several technologies involving the development of malaria vaccines using different formulations with Plasmodium antigens or immunogenic fragments have been reported (U.S. Pat. No. 5,112,749A, US20160038580A1, EP1544211A1, US20190374629A US20040137512, WO2020128031A2). Due to the complexity of the Plasmodium biological cycle, most vaccines reported against malaria have more than one target antigen.

The most advanced candidate to-date is the pre-erythrocytic P. falciparum (RTS, S) vaccine (trade name Mosquirix). The most notable results described for RTS, S vaccine were: (i) 34% efficacy with significant protection against natural P. falciparum infection, (ii) safety and immunogenic in infants, and (iii) a three-dose vaccination with RTS, S was protective against clinical malaria.

The second most advanced stage vaccine is the inactivated sporozoite vaccine from P. falciparum, PfSPZ. In 2002, Sanaria Inc. was created to develop and market a sporozoite-based vaccine. The organization first developed the PfSPZ vaccine in 2003, consisting of an intravenous vaccine with the radiation-attenuated Plasmodium falciparum: parasite.

Finally, there is a pre-erythrocytic malaria vaccine candidate R21/Matrix-M that was developed at the University of Oxford (Oxford, UK) and is currently manufactured by the Serum Institute of India (Pune, India)

US 20160038580 A1 also provides a new nucleotide sequence and other constructs used for the expression of recombinant P. falciparum CS proteins in bacterial cells, such as Escherichia coli. The approach is also used in the AU2004309380B2 invention, which relates to living genetically modified Plasmodium organisms and their use as immuno-effectors for vaccination purposes. The upregulated genes in infective sporozoites 3 and 4 (UIS3 and UIS4) are essential for the early development of the liver stage. This technology provides the first living, genetically modified Plasmodium organisms, the sporozoites UIS3 (−) and UIS4 (−), which infect hepatocytes but are no longer able to establish infections in the blood stage and, therefore, do not lead to disease.

While the development of a live vaccine could raise concerns regarding safety requirements, in addition to scale-up in the vaccine production, the EP1544211A1 invention describes a new P. Falciparum liver sporozoite antigen is called Liver Stage Antigen-5 (LSA-5).

The invention WO2013108272A3 describes a receptor-blocking vaccine based on a combination of new erythrocyte-binding merozoite antigens that includes the PfRH (PfRH1, PfRH2a, PfRH2b, PfRH2b, PfRH4, and PfRH5). Another invention, US20190374629A, provides a vaccine composition in which the PfRH5 antigen triggered antibody production, resulting in at least 50% growth inhibitory activity (GIA) against a plurality of Plasmodium parasite blood-stage genetic strains. The inventors of US20140186402A1 provide an immunogenic composition for its use as a blood-stage malaria vaccine.

The US20080026010 invention describes the administration of a malaria parasite (P. vivax, P. malariae, P. ovale, and P. falciparum) with a modified gene to prevent infection in the host's red blood cells. The P. falciparum depends on the acquisition of purines from the host for its survival in human erythrocytes.

Alternatively, the use of a synthetic malaria vaccine instead of live parasites is described in the U.S. Pat. No. 4,957,738. This invention is a synthetic hybrid protein copolymer used as a human vaccine against the P. falciparum asexual stages.

The invention DK2763694T3 describes a method of producing a cysteine-rich protein (CYRP) vaccine produced in bacteria derived from Pfs48/45 from P. falciparum. The WO2010036293A1 patent also describes the efficient and successful expression of the pre-fertilization antigen Pfs48/45 in high yields and appropriate conformation. A similar approach is described in the CN104736710A and US20150191518A1 inventions. In the CN104736710A patent, the authors used the P. falciparum P47 (Pfs47) or P. vivax P47 (Pfs47) surface antigens. In US20150191518A1, the authors reported a formulation capable of inhibiting the development of P. falciparum inside the mosquito.

The idea to provide an additional immune response to the first, second, and third barriers against the Plasmodium infection was described in invention EP2923709A1. This technology involves new malaria vaccines composed of different recombinant proteins, particularly recombinant fusion proteins comprising several antigens of the P. falciparum from the pre-erythrocytic, blood, and sexual stages. Using a similar approach, CA2910322A1 proposes new recombinant fusion proteins against P. falciparum containing two or more different surface proteins introduced in at least two stages of the parasite's life cycle. Furthermore, the WO2017142843 invention provides polypeptides useful as antigens expressed in both the pre- and erythrocytic stages.

TABLE 1
Patents of malaria vaccines

| Registration Number | Invention Name | Inventors | Target Stage | Target Antigen | Database | Patent Date |
|---|---|---|---|---|---|---|
| U.S. Pat. No. 5,112,749A | Vaccines for the malaria | Brey, | Pre-erythrocytic | Circumsporozoite | USPTO, | 12 May 1992 |
| | circumsporozoite protein | Robert, | stage | recombinant | Espacenet | |
| | | et al. | | proteins | | |
| US20160038580A1 | Soluble recombinant | Dutta, | Pre- | Circumsporozoite | USPTO | 11 Feb. 2016 |
| | P. falciparum circumsporozoite | Sheetij | erythrocytic | recombinant | | |
| | protein, used in | | stage | proteins | | |
| | vaccines, production | | | | | |
| | methods, and uses | | | | | |
| AU2004309380B2 | Live genetically | Stefan, | Pre- | Live Plasmodium | Espacenet | 29 Apr. 2010 |
| | attenuated malaria | Kappe, | erythrocytic | organisms | | |
| | vaccine | et al. | stage | genetically | | |
| | | | | engineered | | |
| AU2013250814B2 | Rodent Plasmodium | Mendes, | Pre- | Live Plasmodium | Espacenet | 2 Mar. 2017 |
| | parasites as platforms | Antonio | erythrocytic | organisms | | |
| | for a whole-organism | Manuel | stage | genetically | | |
| | malaria vaccine | Barbeiro | | engineered | | |
| | | et al. | | | | |
| EP1544211A1 | LSA-5 pre- | Brahimi- | Pre- | P. falciparum liver | Espacenet | 22 Jun. 2005 |
| | erythrocytic stage | Zeghidour, | erythrocytic | sporozoite antigen | | |
| | antigen of | Karima and | stage | | | |
| | P. falciparum, an | an Druilhe, | | | | |
| | immunogenic | Pierre | | | | |
| | composition | | | | | |
| | comprising the antigen | | | | | |
| | and vaccines against | | | | | |
| | malaria | | | | | |
| WO2013108272A3 | Blood-stage malaria | Gaur, | Blood | Merozoite | Patent | 25 Jul. 2013 |
| | vaccine | Deepak, | stage | antigens | scope, | |
| | | et al. | | | Espacenet | |
| US20190374629A | Thermostable variants | Draper, | Blood | Modified | USPTO, | 12 Dec. 2019 |
| | of P. Falciparum | Simon, | stage | homologous | espacenet | |
| | PfRH5 that | et al. | | reticulocyte | | |
| | can be produced in | | | binding | | |
| | bacterial cells | | | | | |
| US20140186402A1 | Blood Stage Malaria | Good, | Blood | Isolated or | USPTO, | 3 Jul. 2014 |
| | Vaccine | Michael | stage | purified | espacenet | |
| | | Spithill, | | merozoites | | |
| | | Terry Lee, | | | | |
| | | Moses | | | | |
| US20080026010 | Use of | Ben | Blood | Attenuated | Patent | 31 Jan. 2008 |
| | conditional Plasmodium | Mamoun | stage | malarial parasite | scope, | |
| | strains lacking | Choukri, | | | Espacenet, | |
| | nutrient transporters in | El Bissati | | | USPTO | |
| | malaria vaccination | Kamal | | | | |
| U.S. Pat. No. 4,957,738 | Protein copolymer | Patarroyo, | Blood | Circumsporozoite | USPTO, | 18 Sep. 1990 |
| | malaria vaccine | Manuel | stage | proteins synthetic | espacenet | |
| US20040137512 | Malaria Plasmodium | Horii, | Blood | serine-repeat | Patent | 15 Jul. 2004 |
| | antigen polypeptide | Toshihiro | stage | antigen of | scope, | |
| | SE36, method of | | | P. falciparum | Espacenet, | |
| | purifying the same and | | | | USPTO, | |
| | vaccine and diagnosis | | | | Latipat | |
| | with the use of the | | | | | |
| | resulting antigen | | | | | |
| DK2763694T3 | Preparation of a | Andersen, | Sexual | Pfs48/45 | espacenet | 16 Apr. 2018 |
| | cysteine-rich protein | Michael | stage | recombinant | | |
| | | TheisenGorm | | protein | | |
| WO2010036293A1 | Malaria vaccine | Kumar, | Sexual | Pfs48/45 | Patent | 3 Jun. 2010 |
| | | Nirbhay | stage | recombinant | scope, | |
| | | Angov, | | protein | Espacenet, | |
| | | Evelina | | | | |
| CN104736710A | Use of p47 from | Barillas- | Sexual | P. falciparum P47 | espacenet | 24 Jun. 2015 |
| | P. falciparum (pfs47) | Mury, | stage | (Pfs47) or | | |
| | or P. vivax (pvs47) as a | Carolina | | P. vivax P47 (Pfs47) | | |
| | vaccine or drug | Veronica | | surface antigens | | |
| | screening target for the | et al. | | | | |
| | inhibition of human | | | | | |
| | malaria transmission | | | | | |
| US20150191518A1 | Novel malaria | Pradel, | Sexual | P. falciparum | USPTO, | 9 Jul. 2015 |
| | transmission-blocking | Gabriele | stage | gliding-associated | espacenet | |
| | vaccines | et al. | | protein 50 | | |
| EP2923709A1 | Multi-component- | Boes, | Various | Recombinant | Espacenet | 20 Sep. 2015 |
| | multistage malaria | Alexander | Stage | fusion proteins | | |
| | vaccine | et al. | Vaccines | | | |
| CA2910322A1 | Novel vaccines against | Boes, | Various | Recombinant | Espacenet | 20 Oct. 2014 |
| | apicomplexan | Alexander | Stage | fusion proteins | | |
| | pathogens | et al. | Vaccines | | | |
| EP2992895 | Three-component- | Fischer, | Various | Recombinant | Espacenet | 9 Mar. 2016 |
| | multistage malaria | Rainer | Stage | fusion proteins | | |
| | vaccine | et al. | Vaccines | | | |
| WO2017142843 | Novel antigen for | AGUIAR, | Various | Recombinant | Patent | 24 Aug. 2017 |
| | use in malaria | Joao Carlos | Stage | fusion proteins | scope | |
| | vaccine | | Vaccines | | | |

### DETAILS OF THE INVENTION

Table 2 lists the invention process of selecting the most likely antigen sources.

TABLE 2
mRNA vaccine design of the invention (T-cell and B-cell epitopes)

| Protein | Peptide/Protein | Sequence |
|---|---|---|
| Circumsporozoite protein (CS) | Entire Protein | MMRKLAILSVSSFLFVEALFQE |
| Q5R2L8•Q5R2L8 PLAFA | | YQCYGSSSNTRVLNELNYDNA |
| Taxonomy-Plasmodium | | GTNLYNELEMNYYGKQENWYS |
| falciparum | | LKKNSRSLGENDDGNNNNGDN |
| | | GREGKDEDKRDGNNEDNEKLR |
| | | KPKHKKLKQPGDGNPDPNANP |
| | | NVDPNANPNVDPNANPNVDPN |
| | | ANPNANPNANPNANPNANPNA |
| | | NPNANPNANPNANPNANPNAN |
| | | PNANPNANPNANPNANPNVDP |
| | | NANPNANPNANPNANPNANPN |
| | | ANPNANPNANPNANPNANPNA |
| | | NPNANPNANPNANPNANPNAN |
| | | PNANPNANPNANPNKNNQGNG |
| | | QGHNMPNDPNRNVDENANAN |
| | | NAVKNNNNEEPSDKHIEQYLKK |
| | | IKNSISTEWSPCSVTCGNGIQVRI |
| | | KPGSANKPKDELDYENDIEKKI |
| | | CKMEKCSSVFNVVNSSIGLIMV |
| | | LSFLFLN |
| Merozoite Surface Protein 1 | Peptide 20-39 | VTHESYQELVKKLEALEDAV |
| (MSP1): | Peptide 44-63 | GLFHKEKMILNEEEITTKGA |
| P50495•MSP1_PLAFP | Peptide 1448-1461 | QEGISYYEKVLAKY |
| (Uganda): | Peptide 1505-1519 | FLTNIETLYNNLVNK |
| Taxonomy-Plasmodium | Peptide 1711-1725 | GISFLLILMLILYSF |
| falciparum (isolate Palo Alto/ | | |
| Uganda) | | |
| Apical Membrane Antigen 1 | Peptide 189-206 | LMSPMTLDEMRHFYKDNK |
| (AMA1): P22621• | Peptide 345-360 | SASDQPKQYEQHLTDY |
| AMA1 PLAFF•New Guinea: | Peptide 547-555 | IIIASSAAV |
| Taxonomy-Plasmodium | Peptide 367-380 | GFKNKNASMIKSAFL |
| falciparum (isolate FC27/ | | |
| Papua New Guinea) | | |
| Erythrocyte Binding Antigens | Peptide 285-303 | VWECKNPYILSTKDVCVPP |
| (EBAs) A0A023ZZ81•1_PLAFA: | Peptide 328-342 | DKNLLMIKEHILAIA |
| | Peptide 364-374 | DIIGGTDYW |
| Taxonomy-Plasmodium | Peptide 311-325 | GNIDRIYDKNLLMIK |
| falciparum Vietnam Oak-Knoll | | |
| (FVO) (no rank | | |
| Rhoptry Neck Proteins (RONs): | Peptide 1464-1475 | KYTTPVFPMSAP |
| B6KV60•RON2 TOXGM: | Peptide 286-300 | PSGIRVYATPPAPRP |
| Reticulocyte-binding protein | Peptide 1001-1015 | IAKRVHQSKNLLRRA |
| homolog 5: Taxonomy: | | |
| Toxoplasma gondii (strain | | |
| ATCC 50611/Me49) | | |
| Rhoptry neck protein 2: | Peptide 729-748 | YGRTRNKRYMHRNPGEKYKG |
| IOJ1F2•IOJ1F2_PLAVI | | |
| Taxonomy-Plasmodium vivax | | |
| IOJ114.IOJ1I4_PLAVI: | Peptide 40-54 | RGPVNYHFSNYMNLD |
| Rhoptry neck protein 2 | | |
| Taxonomy-Plasmodium vivax | | |
| PfRh Proteins: Q8IFM5• | Peptide 249-263 | DDSYRYDISEEIDDK |
| RH5_PLAF7: | Peptide 209-217 | FIKKINETY |
| Taxonomy-Plasmodium | Peptide 366-380 | KLILSVKSKNLNKDL |
| falciparum Vietnam Oak-Knoll | Peptide 6-20 | KKLILTIIYIHLFIL |
| (FVO) (no rank) | | |

| | |
|---|---|
| Final Peptide Sequence with | MMRKLAILSVSSFLFVEALFQEYQCYGSSSNTRVLN |
| Linker Attached (One protein | ELNYDNAGTNLYNELEMNYYGKQENWYSLKKNSR |
| and 22 Peptides) from 8 | SLGENDDGNNNNGDNGREGKDEDKRDGNNEDNEK |
| Proteins (dash to show linking | LRKPKHKKLKQPGDGNPDPNANPNVDPNANPNVDP |
| of GSGSGSGS linker | NANPNVDPNANPNANPNANPNANPNANPNANPNA |
| | NPNANPNANPNANPNANPNANPNANPNANPNANPN |
| | VDPNANPNANPNANPNANPNANPNANPNANPNANP |
| | NANPNANPNANPNANPNANPNANPNANPNANPNA |
| | NPNANPNANPNKNNQGNGQGHNMPNDPNRNVDEN |
| | ANANNAVKNNNNEEPSDKHIEQYLKKIKNSISTEWS |
| | PCSVTCGNGIQVRIKPGSANKPKDELDYENDIEKKIC |
| | KMEKCSSVFNVVNSSIGLIMVLSFLFLNGSGSGSGS |
| | VTHESYQELVKKLEALEDAVGSGSGSGSGLFHKEK |
| | MILNEEEITTKGAGSGSGSGSQEGISYYEKVLAKYG |
| | SGSGSGSFLTNIETLYNNLVNKGSGSGSGSGISFLLI |
| | LMLILYSFGSGSGSGSLMSPMTLDEMRHFYKDNKG |
| | SGSGSGSSASDQPKQYEQHLTDYGSGSGSGSIIIASS |
| | AAVGSGSGSGSGFKNKNASMIKSAFLGSGSGSGSV |
| | WECKNPYILSTKDVCVPPGSGSGSGSDKNLLMIKEH |
| | ILAIAGSGSGSGSDIIGGTDYWGSGSGSGSGNIDRIY |
| | DKNLLMIKGSGSGSGSKYTTPVFPMSAPGSGSGSGS |
| | PSGIRVYATPPAPRPGSGSGSGSYGRTRNKRYMHRN |
| | PGEKYKGGSGSGSGSRGPVNYHFSNYMNLDGSGS |
| | GSGSIAKRVHQSKNLLRRAGSGSGSGSDDSYRYDIS |
| | EEIDDKGSGSGSGSFIKKINETYGSGSGSGSKLILSV |
| | KSKNLNKDLGSGSGSGSKKLILTIIYIHLFIL |
| DNA Sequence (2757) | atgatgcgcaaactggcgattctgagcgtgagcagctttctgtttgtggaagcgctg |
| | tttcaggaatatcagtgctatggcagcagcagcaacacccgcgtgctgaacgaact |
| | gaactatgataacgcgggcaccaacctgtataacgaactggaaatgaactattatg |
| | gcaaacaggaaaactggtatagcctgaaaaaaaacagccgcagcctgggcgaa |
| | aacgatgatggcaacaacaacaacggcgataacggccgcgaaggcaaagatga |
| | agataaacgcgatggcaacaacgaagataacgaaaaactgcgcaaaccgaaac |
| | ataaaaaactgaaacagccgggcgatggcaacccggatccgaacgcgaacccg |
| | aacgtggatccgaacgcgaacccgaacgtggatccgaacgcgaacccgaacgt |
| | ggatccgaacgcgaacccgaacgcgaacccgaacgcgaacccgaacgcgaac |
| | ccgaacgcgaacccgaacgcgaacccgaacgcgaacccgaacgcgaacccga |
| | acgcgaacccgaacgcgaacccgaacgcgaacccgaacgcgaacccgaacgc |
| | gaacccgaacgcgaacccgaacgcgaacccgaacgtggatccgaacgcgaac |
| | ccgaacgcgaacccgaacgcgaacccgaacgcgaacccgaacgcgaacccga |
| | acgcgaacccgaacgcgaacccgaacgcgaacccgaacgcgaacccgaacgc |
| | gaacccgaacgcgaacccgaacgcgaacccgaacgcgaacccgaacgcgaac |
| | ccgaacgcgaacccgaacgcgaacccgaacgcgaacccgaacgcgaacccga |
| | acgcgaacccgaacaaaaacaaccagggcaacggccagggccataacatgcc |
| | gaacgatccgaaccgcaacgtggatgaaaacgcgaacgcgaacaacgcggtga |
| | aaaacaacaacaacgaagaaccgagcgataaacatattgaacagtatctgaaaaa |
| | aattaaaaacagcattagcaccgaatggagcccgtgcagcgtgacctgcggcaa |
| | cggcattcaggtgcgcattaaaccgggcagcgcgaacaaaccgaaagatgaact |
| | ggattatgaaaacgatattgaaaaaaaaatttgcaaaatggaaaaatgcagcagcg |
| | tgtttaacgtggtgaacagcagcattggcctgattatggtgctgagctttctgtttctg |
| | aacggcagcggcagcggcagcggcagcgtgacccatgaaagctatcaggaact |
| | ggtgaaaaaactggaagcgctggaagatgcggtgggcagcggcagcggcagc |
| | ggcagcggcctgtttcataaagaaaaaatgattctgaacgaagaagaaattaccac |
| | caaaggcgcgggcagcggcagcggcagcggcagccaggaaggcattagctat |
| | tatgaaaaagtgctggcgaaatatggcagcggcagcggcagcggcagctttctga |
| | ccaacattgaaaccctgtataacaacctggtgaacaaaggcagcggcagcggca |
| | gcggcagcggcattagctttctgctgattctgatgctgattctgtatagctttggcagc |
| | ggcagcggcagcggcagcctgatgagcccgatgaccctggatgaaatgcgcca |
| | tttttataaagataacaaaggcagcggcagcggcagcggcagcagcgcgagcga |
| | tcagccgaaacagtatgaacagcatctgaccgattatggcagcggcagcggcag |
| | cggcagcattattattgcgagcagcgcggcggtgggcagcggcagcggcagcg |
| | gcagcggctttaaaaacaaaaacgcgagcatgattaaaagcgcgtttctgggcag |
| | cggcagcggcagcggcagcgtgtgggaatgcaaaaacccgtatattctgagcac |
| | caaagatgtgtgcgtgccgccgggcagcggcagcggcagcggcagcgataaa |
| | aacctgctgatgattaaagaacatattctggcgattgcgggcagcggcagcggca |
| | gcggcagcgatattattggcggcaccgattattggggcagcggcagcggcagcg |
| | gcagcggcaacattgatcgcatttatgataaaaacctgctgatgattaaaggcagc |
| | ggcagcggcagcggcagcaaatataccaccccggtgtttccgatgagcgcgccg |
| | ggcagcggcagcggcagcggcagcccgagcggcattcgcgtgtatgcgaccc |
| | cgccggcgccgcgcccgggcagcggcagcggcagcggcagctatggccgca |
| | cccgcaacaaacgctatatgcatcgcaacccgggcgaaaaatataaaggcggca |
| | gcggcagcggcagcggcagccgcggcccggtgaactatcattttagcaactatat |
| | gaacctggatggcagcggcagcggcagcggcagcattgcgaaacgcgtgcatc |
| | agagcaaaaacctgctgcgccgcgcgggcagcggcagcggcagcggcagcg |
| | atgatagctatcgctatgatattagcgaagaaattgatgataaaggcagcggcagc |
| | ggcagcggcagctttattaaaaaaattaacgaaacctatggcagcggcagcggca |
| | gcggcagcaaactgattctgagcgtgaaaagcaaaaacctgaacaaagatctgg |
| | gcagcggcagcggcagcggcagcaaaaaactgattctgaccattatttatattcat |
| | ctgtttattctg |
| RNA Sequence (2757) | AUGAUGCGCAAACUGGCGAUUCUGAGCGUGAGC |
| | AGCUUUCUGUUUGUGGAAGCGCUGUUUCAGGAA |
| | UAUCAGUGCUAUGGCAGCAGCAGCAACACCCGC |
| | GUGCUGAACGAACUGAACUAUGAUAACGCGGGC |
| | ACCAACCUGUAUAACGAACUGGAAAUGAACUAU |
| | UAUGGCAAACAGGAAAACUGGUAUAGCCUGAAA |
| | AAAAACAGCCGCAGCCUGGGCGAAAACGAUGAU |
| | GGCAACAACAACAACGGCGAUAACGGCCGCGAA |
| | GGCAAAGAUGAAGAUAAACGCGAUGGCAACAAC |
| | GAAGAUAACGAAAAACUGCGCAAACCGAAACAU |
| | AAAAAACUGAAACAGCCGGGCGAUGGCAACCCG |
| | GAUCCGAACGCGAACCCGAACGUGGAUCCGAAC |
| | GCGAACCCGAACGUGGAUCCGAACGCGAACCCG |
| | AACGUGGAUCCGAACGCGAACCCGAACGCGAAC |
| | CCGAACGCGAACCCGAACGCGAACCCGAACGCGA |
| | ACCCGAACGCGAACCCGAACGCGAACCCGAACGC |
| | GAACCCGAACGCGAACCCGAACGCGAACCCGAAC |
| | GCGAACCCGAACGCGAACCCGAACGCGAACCCGA |
| | ACGCGAACCCGAACGCGAACCCGAACGUGGAUC |
| | CGAACGCGAACCCGAACGCGAACCCGAACGCGA |
| | ACCCGAACGCGAACCCGAACGCGAACCCGAACGC |
| | GAACCCGAACGCGAACCCGAACGCGAACCCGAAC |
| | GCGAACCCGAACGCGAACCCGAACGCGAACCCGA |
| | ACGCGAACCCGAACGCGAACCCGAACGCGAACCC |
| | GAACGCGAACCCGAACGCGAACCCGAACGCGAA |
| | CCCGAACGCGAACCCGAACGCGAACCCGAACAAA |
| | AACAACCAGGGCAACGGCCAGGGCCAUAACAUG |
| | CCGAACGAUCCGAACCGCAACGUGGAUGAAAAC |
| | GCGAACGCGAACAACGCGGUGAAAAACAACAAC |
| | AACGAAGAACCGAGCGAUAAACAUAUUGAACAG |
| | UAUCUGAAAAAAAUUAAAAACAGCAUUAGCACC |
| | GAAUGGAGCCCGUGCAGCGUGACCUGCGGCAAC |
| | GGCAUUCAGGUGCGCAUUAAACCGGGCAGCGCG |
| | AACAAACCGAAAGAUGAACUGGAUUAUGAAAAC |
| | GAUAUUGAAAAAAAAAUUUGCAAAAUGGAAAAA |
| | UGCAGCAGCGUGUUUAACGUGGUGAACAGCAGC |
| | AUUGGCCUGAUUAUGGUGCUGAGCUUUCUGUUU |
| | CUGAACGGCAGCGGCAGCGGCAGCGGCAGCGUG |
| | ACCCAUGAAAGCUAUCAGGAACUGGUGAAAAAA |
| | CUGGAAGCGCUGGAAGAUGCGGUGGGCAGCGGC |
| | AGCGGCAGCGGCAGCGGCCUGUUUCAUAAAGAA |
| | AAAAUGAUUCUGAACGAAGAAGAAAUUACCACC |
| | AAAGGCGCGGGCAGCGGCAGCGGCAGCGGCAGC |
| | CAGGAAGGCAUUAGCUAUUAUGAAAAAGUGCUG |
| | GCGAAAUAUGGCAGCGGCAGCGGCAGCGGCAGC |
| | UUUCUGACCAACAUUGAAACCCUGUAUAACAAC |
| | CUGGUGAACAAAGGCAGCGGCAGCGGCAGCGGC |
| | AGCGGCAUUAGCUUUCUGCUGAUUCUGAUGCUG |
| | AUUCUGUAUAGCUUUGGCAGCGGCAGCGGCAGC |
| | GGCAGCCUGAUGAGCCCGAUGACCCUGGAUGAA |
| | AUGCGCCAUUUUUAUAAAGAUAACAAAGGCAGC |
| | GGCAGCGGCAGCGGCAGCAGCGCGAGCGAUCAG |
| | CCGAAACAGUAUGAACAGCAUCUGACCGAUUAU |
| | GGCAGCGGCAGCGGCAGCGGCAGCAUUAUUAUU |
| | GCGAGCAGCGCGGCGGUGGGCAGCGGCAGCGGC |
| | AGCGGCAGCGGCUUUAAAAACAAAAACGCGAGC |
| | AUGAUUAAAAGCGCGUUUCUGGGCAGCGGCAGC |
| | GGCAGCGGCAGCGUGUGGGAAUGCAAAAACCCG |
| | UAUAUUCUGAGCACCAAAGAUGUGUGCGUGCCG |
| | CCGGGCAGCGGCAGCGGCAGCGGCAGCGAUAAA |
| | AACCUGCUGAUGAUUAAAGAACAUAUUCUGGCG |
| | AUUGCGGGCAGCGGCAGCGGCAGCGGCAGCGAU |
| | AUUAUUGGCGGCACCGAUUAUUGGGGCAGCGGC |
| | AGCGGCAGCGGCAGCGGCAACAUUGAUCGCAUU |
| | UAUGAUAAAAACCUGCUGAUGAUUAAAGGCAGC |
| | GGCAGCGGCAGCGGCAGCAAAUAUACCACCCCG |
| | GUGUUUCCGAUGAGCGCGCCGGGCAGCGGCAGC |
| | GGCAGCGGCAGCCCGAGCGGCAUUCGCGUGUAU |
| | GCGACCCCGCCGGCGCCGCGCCCGGGCAGCGGCA |
| | GCGGCAGCGGCAGCUAUGGCCGCACCCGCAACA |
| | AACGCUAUAUGCAUCGCAACCCGGGCGAAAAAU |
| | AUAAAGGCGGCAGCGGCAGCGGCAGCGGCAGCC |
| | GCGGCCCGGUGAACUAUCAUUUUAGCAACUAUA |
| | UGAACCUGGAUGGCAGCGGCAGCGGCAGCGGCA |
| | GCAUUGCGAAACGCGUGCAUCAGAGCAAAAACC |
| | UGCUGCGCCGCGCGGGCAGCGGCAGCGGCAGCG |
| | GCAGCGAUGAUAGCUAUCGCUAUGAUAUUAGCG |
| | AAGAAAUUGAUGAUAAAGGCAGCGGCAGCGGCA |
| | GCGGCAGCUUUAUUAAAAAAAUUAACGAAACCU |
| | AUGGCAGCGGCAGCGGCAGCGGCAGCAAACUGA |
| | UUCUGAGCGUGAAAAGCAAAAACCUGAACAAAG |
| | AUCUGGGCAGCGGCAGCGGCAGCGGCAGCAAAA |
| | AACUGAUUCUGACCAUUAUUUAUAUUCAUCUGU |
| | UUAUUCUG |
| Protein Sequence Recheck | MMRKLAILSVSSFLFVEALFQEYQCYGSSSNTRVLN |
| (919) | ELNYDNAGTNLYNELEMNYYGKQENWYSLKKNSR |
| | SLGENDDGNNNNGDNGREGKDEDKRDGNNEDNEK |
| | LRKPKHKKLKQPGDGNPDPNANPNVDPNANPNVDP |
| | NANPNVDPNANPNANPNANPNANPNANPNANPNA |
| | NPNANPNANPNANPNANPNANPNANPNANPNANPN |
| | VDPNANPNANPNANPNANPNANPNANPNANPNANP |
| | NANPNANPNANPNANPNANPNANPNANPNANPNA |
| | NPNANPNANPNKNNQGNGQGHNMPNDPNRNVDEN |
| | ANANNAVKNNNNEEPSDKHIEQYLKKIKNSISTEWS |
| | PCSVTCGNGIQVRIKPGSANKPKDELDYENDIEKKIC |
| | KMEKCSSVFNVVNSSIGLIMVLSFLFLNGSGSGSGS |
| | VTHESYQELVKKLEALEDAVGSGSGSGSGLFHKEK |
| | MILNEEEITTKGAGSGSGSGSQEGISYYEKVLAKYG |
| | SGSGSGSFLTNIETLYNNLVNKGSGSGSGSGISFLLI |
| | LMLILYSFGSGSGSGSLMSPMTLDEMRHFYKDNKG |
| | SGSGSGSSASDQPKQYEQHLTDYGSGSGSGSIIIASS |
| | AAVGSGSGSGSGFKNKNASMIKSAFLGSGSGSGSV |
| | WECKNPYILSTKDVCVPPGSGSGSGSDKNLLMIKEH |
| | ILAIAGSGSGSGSDIIGGTDYWGSGSGSGSGNIDRIY |
| | DKNLLMIKGSGSGSGSKYTTPVFPMSAPGSGSGSGS |
| | PSGIRVYATPPAPRPGSGSGSGSYGRTRNKRYMHRN |
| | PGEKYKGGSGSGSGSRGPVNYHFSNYMNLDGSGS |
| | GSGSIAKRVHQSKNLLRRAGSGSGSGSDDSYRYDIS |
| | EEIDDKGSGSGSGSFIKKINETYGSGSGSGSKLILSV |
| | KSKNLNKDLGSGSGSGSKKLILTIIYIHLFIL |
| ORF Sequence with uridine is | AΨGAΨGCGCAAACΨGGCGAΨΨCΨGAGCGΨGAGC |
| replaced with Y = 1-methyl-3′- | AGCΨΨΨCΨGΨΨΨGΨGGAAGCGCΨGΨΨΨCAGGAA |
| pseudouridylyl | ΨAΨCAGΨGCΨAΨGGCAGCAGCAGCAACACCCGC |
| | GΨGCΨGAACGAACΨGAACΨAΨGAΨAACGCGGGC |
| | ACCAACCΨGΨAΨAACGAACΨGGAAAΨGAACΨAΨ |
| | ΨAΨGGCAAACAGGAAAACΨGGΨAΨAGCCΨGAAA |
| | AAAAACAGCCGCAGCCΨGGGCGAAAACGAΨGAΨ |
| | GGCAACAACAACAACGGCGAΨAACGGCCGCGAA |
| | GGCAAAGAΨGAAGAΨAAACGCGAΨGGCAACAAC |
| | GAAGAΨAACGAAAAACΨGCGCAAACCGAAACAΨ |
| | AAAAAACΨGAAACAGCCGGGCGAΨGGCAACCCG |
| | GAΨCCGAACGCGAACCCGAACGΨGGAΨCCGAAC |
| | GCGAACCCGAACGΨGGAΨCCGAACGCGAACCCG |
| | AACGΨGGAΨCCGAACGCGAACCCGAACGCGAAC |
| | CCGAACGCGAACCCGAACGCGAACCCGAACGCGA |
| | ACCCGAACGCGAACCCGAACGCGAACCCGAACGC |
| | GAACCCGAACGCGAACCCGAACGCGAACCCGAAC |
| | GCGAACCCGAACGCGAACCCGAACGCGAACCCGA |
| | ACGCGAACCCGAACGCGAACCCGAACGΨGGAΨC |
| | CGAACGCGAACCCGAACGCGAACCCGAACGCGA |
| | ACCCGAACGCGAACCCGAACGCGAACCCGAACGC |
| | GAACCCGAACGCGAACCCGAACGCGAACCCGAAC |
| | GCGAACCCGAACGCGAACCCGAACGCGAACCCGA |
| | ACGCGAACCCGAACGCGAACCCGAACGCGAACCC |
| | GAACGCGAACCCGAACGCGAACCCGAACGCGAA |
| | CCCGAACGCGAACCCGAACGCGAACCCGAACAAA |
| | AACAACCAGGGCAACGGCCAGGGCCAΨAACAΨG |
| | CCGAACGAΨCCGAACCGCAACGΨGGAΨGAAAAC |
| | GCGAACGCGAACAACGCGGΨGAAAAACAACAAC |
| | AACGAAGAACCGAGCGAΨAAACAΨAΨΨGAACAG |
| | ΨAΨCΨGAAAAAAAΨΨAAAAACAGCAΨΨAGCACC |
| | GAAΨGGAGCCCGΨGCAGCGΨGACCΨGCGGCAAC |
| | GGCAΨΨCAGGΨGCGCAΨΨAAACCGGGCAGCGCG |
| | AACAAACCGAAAGAΨGAACΨGGAΨΨAΨGAAAAC |
| | GAΨAΨΨGAAAAAAAAAΨΨΨGCAAAAΨGGAAAA |
| | AΨGCAGCAGCGΨGΨΨΨAACGΨGGΨGAACAGCAG |
| | CAΨΨGGCCΨGAΨΨAΨGGΨGCΨGAGCΨΨΨCΨGΨΨ |
| | ΨCΨGAACGGCAGCGGCAGCGGCAGCGGCAGCGΨ |
| | GACCCAΨGAAAGCΨAΨCAGGAACΨGGΨGAAAAA |
| | ACΨGGAAGCGCΨGGAAGAΨGCGGΨGGGCAGCGG |
| | CAGCGGCAGCGGCAGCGGCCΨGΨΨΨCAΨAAAGA |
| | AAAAAΨGAΨΨCΨGAACGAAGAAGAAAΨΨACCAC |
| | CAAAGGCGCGGGCAGCGGCAGCGGCAGCGGCAG |
| | CCAGGAAGGCAΨΨAGCΨAΨΨAΨGAAAAAGΨGCΨ |
| | GGCGAAAΨAΨGGCAGCGGCAGCGGCAGCGGCAG |
| | CΨΨΨCΨGACCAACAΨΨGAAACCCΨGΨAΨAACAA |
| | CCΨGGΨGAACAAAGGCAGCGGCAGCGGCAGCGG |
| | CAGCGGCAΨΨAGCΨΨΨCΨGCΨGAΨΨCΨGAΨGCΨ |
| | GAΨΨCΨGΨAΨAGCΨΨΨGGCAGCGGCAGCGGCAG |
| | CGGCAGCCΨGAΨGAGCCCGAΨGACCCΨGGAΨGA |
| | AAΨGCGCCAΨΨΨΨΨAΨAAAGAΨAACAAAGGCAG |
| | CGGCAGCGGCAGCGGCAGCAGCGCGAGCGAΨCA |
| | GCCGAAACAGΨAΨGAACAGCAΨCΨGACCGAΨΨA |
| | ΨGGCAGCGGCAGCGGCAGCGGCAGCAΨΨAΨΨAΨ |
| | ΨGCGAGCAGCGCGGCGGΨGGGCAGCGGCAGCGG |
| | CAGCGGCAGCGGCΨΨΨAAAAACAAAAACGCGAG |
| | CAΨGAΨΨAAAAGCGCGΨΨΨCΨGGGCAGCGGCAG |
| | CGGCAGCGGCAGCGΨGΨGGGAAΨGCAAAAACCC |
| | GΨAΨAΨΨCΨGAGCACCAAAGAΨGΨGΨGCGΨGCC |
| | GCCGGGCAGCGGCAGCGGCAGCGGCAGCGAΨAA |
| | AAACCΨGCΨGAΨGAΨΨAAAGAACAΨAΨΨCΨGGC |
| | GAΨΨGCGGGCAGCGGCAGCGGCAGCGGCAGCGA |
| | ΨAΨΨAΨΨGGCGGCACCGAΨΨAΨΨGGGGCAGCGG |
| | CAGCGGCAGCGGCAGCGGCAACAΨΨGAΨCGCAΨ |
| | ΨΨAΨGAΨAAAAACCΨGCΨGAΨGAΨΨAAAGGCAG |
| | CGGCAGCGGCAGCGGCAGCAAAΨAΨACCACCCC |
| | GGΨGΨΨΨCCGAΨGAGCGCGCCGGGCAGCGGCAG |
| | CGGCAGCGGCAGCCCGAGCGGCAΨΨCGCGΨGΨA |
| | ΨGCGACCCCGCCGGCGCCGCGCCCGGGCAGCGGC |
| | AGCGGCAGCGGCAGCΨAΨGGCCGCACCCGCAAC |
| | AAACGCΨAΨAΨGCAΨCGCAACCCGGGCGAAAAA |
| | ΨAΨAAAGGCGGCAGCGGCAGCGGCAGCGGCAGC |
| | CGCGGCCCGGΨGAACΨAΨCAΨΨΨΨAGCAACΨAΨ |
| | AΨGAACCΨGGAΨGGCAGCGGCAGCGGCAGCGGC |
| | AGCAΨΨGCGAAACGCGΨGCAΨCAGAGCAAAAAC |
| | CΨGCΨGCGCCGCGCGGGCAGCGGCAGCGGCAGC |
| | GGCAGCGAΨGAΨAGCΨAΨCGCΨAΨGAΨAΨΨAGC |
| | GAAGAAAΨΨGAΨGAΨAAAGGCAGCGGCAGCGGC |
| | AGCGGCAGCΨΨΨAΨΨAAAAAAAΨΨAACGAAACC |
| | ΨAΨGGCAGCGGCAGCGGCAGCGGCAGCAAACΨG |
| | AΨΨCΨGAGCGΨGAAAAGCAAAAACCΨGAACAAA |
| | GAΨCΨGGGCAGCGGCAGCGGCAGCGGCAGCAAA |
| | AAACΨGAΨΨCΨGACCAΨΨAΨΨΨAΨAΨΨCAΨCΨG |
| | ΨΨΨAΨΨCΨG |

Table 3 lists the mRNA structure and its components.

TABLE 3
Vaccine Structure for Malaria Vaccine Antigen Production.

| Element | Description | Position |
|---|---|---|
| cap | A modified 5′-cap1 structure (m7G + m3′-5′-ppp-5′-Am): | 1-2 |
| | GA | |
| 5′-UTR | The 5′-untranslated region derived from human alpha- | 3-54 |
| | globin RNA with an optimized Kozak sequence. | |
| | GAAΨAAACΨAGΨAΨΨCΨΨCΨGGΨCCCCACAGACΨCAG | |
| | A GAGAACCCGC CACC | |
| Signal | S glycoprotein signal peptide (extended leader | 55-102 |
| Peptide | sequence) guides translocation of the nascent | |
| | polypeptide chain into the endoplasmic | |
| | reticulum: | |
| | AΨGΨΨCGΨGΨΨCCΨGGΨGCΨGCΨGCCΨCΨGGΨGΨCCA | |
| | GCCAGΨGΨG ΨG | |
| Coding | Codon-optimized sequence: | 103-2859 |
| Region | AΨGAΨGCGCAAACΨGGCGAΨΨCΨGAGCGΨGAGCAGCΨ | |
| (stop | ΨΨCΨGΨΨΨGΨGGAAGCGCΨGΨΨΨCAGGAAΨAΨCAGΨG | |
| codons | CΨAΨGGCAGCAGCAGCAACACCCGCGΨGCΨGAACGAAC | |
| under- | ΨGAACΨAΨGAΨAACGCGGGCACCAACCΨGΨAΨAACGA | |
| lined) | ACΨGGAAAΨGAACΨAΨΨAΨGGCAAACAGGAAAACΨGG | |
| | ΨAΨAGCCΨGAAAAAAAACAGCCGCAGCCΨGGGCGAAA | |
| | ACGAΨGAΨGGCAACAACAACAACGGCGAΨAACGGCCG | |
| | CGAAGGCAAAGAΨGAAGAΨAAACGCGAΨGGCAACAAC | |
| | GAAGAΨAACGAAAAACΨGCGCAAACCGAAACAΨAAAA | |
| | AACΨGAAACAGCCGGGCGAΨGGCAACCCGGAΨCCGAAC | |
| | GCGAACCCGAACGΨGGAΨCCGAACGCGAACCCGAACGΨ | |
| | GGAΨCCGAACGCGAACCCGAACGΨGGAΨCCGAACGCGA | |
| | ACCCGAACGCGAACCCGAACGCGAACCCGAACGCGAAC | |
| | CCGAACGCGAACCCGAACGCGAACCCGAACGCGAACCC | |
| | GAACGCGAACCCGAACGCGAACCCGAACGCGAACCCGA | |
| | ACGCGAACCCGAACGCGAACCCGAACGCGAACCCGAAC | |
| | GCGAACCCGAACGCGAACCCGAACGΨGGAΨCCGAACGC | |
| | GAACCCGAACGCGAACCCGAACGCGAACCCGAACGCGA | |
| | ACCCGAACGCGAACCCGAACGCGAACCCGAACGCGAAC | |
| | CCGAACGCGAACCCGAACGCGAACCCGAACGCGAACCC | |
| | GAACGCGAACCCGAACGCGAACCCGAACGCGAACCCGA | |
| | ACGCGAACCCGAACGCGAACCCGAACGCGAACCCGAAC | |
| | GCGAACCCGAACGCGAACCCGAACGCGAACCCGAACAA | |
| | AAACAACCAGGGCAACGGCCAGGGCCAΨAACAΨGCCGA | |
| | ACGAΨCCGAACCGCAACGΨGGAΨGAAAACGCGAACGC | |
| | GAACAACGCGGΨGAAAAACAACAACAACGAAGAACCG | |
| | AGCGAΨAAACAΨAΨΨGAACAGΨAΨCΨGAAAAAAAΨΨA | |
| | AAAACAGCAΨΨAGCACCGAAΨGGAGCCCGΨGCAGCGΨ | |
| | GACCΨGCGGCAACGGCAΨΨCAGGΨGCGCAΨΨAAACCG | |
| | GGCAGCGCGAACAAACCGAAAGAΨGAACΨGGAΨΨAΨG | |
| | AAAACGAΨAΨΨGAAAAAAAAAΨΨΨGCAAAAΨGGAAAA | |
| | AΨGCAGCAGCGΨGΨΨΨAACGΨGGΨGAACAGCAGCAΨΨ | |
| | GGCCΨGAΨΨAΨGGΨGCΨGAGCΨΨΨCΨGΨΨΨCΨGAACG | |
| | GCAGCGGCAGCGGCAGCGΨGACCCAΨGAAAGCΨAΨCA | |
| | GGAACΨGGΨGAAAAAACΨGGAAGCGCΨGGAAGAΨGCG | |
| | GΨGGGCAGCGGCAGCGGCAGCGGCAGCGGCCΨGΨΨΨC | |
| | AΨAAAGAAAAAAΨGAΨΨCΨGAACGAAGAAGAAAΨΨAC | |
| | CACCAAAGGCGCGGGCAGCGGCAGCGGCAGCGGCAGCC | |
| | AGGAAGGCAΨΨAGΨCAΨΨAΨGAAAAAGΨGCΨGGCGAA | |
| | AΨAΨGGCAGCGGCAGCGGCAGCGGCAGCΨΨΨCΨGACC | |
| | AACAΨΨGAAACCCΨGΨAΨAACAACCΨGGΨGAACAAAG | |
| | GCAGCGGCAGCGGCAGCGGCAGCGGCAΨΨAGCΨΨΨCΨ | |
| | GCΨGAΨΨCΨGAΨGCΨGAΨΨCΨGΨAΨAGCΨΨΨGGCAGC | |
| | GGCAGCGGCAGCGGCAGCCΨGAΨGAGCCCGAΨGACCCΨ | |
| | GGAΨGAAAΨGCGCCAΨΨΨΨΨAΨAAAGAΨAACAAAGGC | |
| | AGCGGCAGCGGCAGCGGCAGCAGCGCGAGCGAΨCAGCC | |
| | GAAACAGΨAΨGAACAGCAΨCΨGACCGAΨΨAΨGGCAGC | |
| | GGCAGCGGCAGCGGCAGCAΨΨAΨΨAΨΨGCGAGCAGCG | |
| | CGGCGGΨGGGCAGCGGCAGCGGCAGCGGCAGCGGCΨΨ | |
| | ΨAAAAACAAAAACGCGAGCAΨGAΨΨAAAAGCGCGΨΨΨ | |
| | CΨGGGCAGCGGCAGCGGCAGCGGCAGCGΨGΨGGGAAΨ | |
| | GCAAAAACCCGΨAΨAΨΨCΨGAGCACCAAAGAΨGΨGΨG | |
| | CGΨGCCGCCGGGCAGCGGCAGCGGCAGCGGCAGCGAΨA | |
| | AAAACCΨGCΨGAΨGAΨΨAAAGAACAΨAΨΨCΨGGCGAΨ | |
| | ΨGCGGGCAGCGGCAGCGGCAGCGGCAGCGAΨAΨΨAΨΨ | |
| | GGCGGCACCGAΨΨAΨΨGGGGCAGCGGCAGCGGCAGCG | |
| | GCAGCGGCAACAΨΨGAΨCGCAΨΨΨAΨGAΨAAAAACCΨ | |
| | GCΨGAΨGAΨΨAAAGGCAGCGGCAGCGGCAGCGGCAGC | |
| | AAAΨAΨACCACCCCGGΨGΨΨΨCCGAΨGAGCGCGCCGG | |
| | GCAGCGGCAGCGGCAGCGGCAGCCCGAGCGGCAΨΨCGC | |
| | GΨGΨAΨGCGACCCCGCCGGCGCCGCGCCCGGGCAGCGG | |
| | CAGCGGCAGCGGCAGCΨAΨGGCCGCACCCGCAACAAAC | |
| | GCΨAΨAΨGCAΨCGCAACCCGGGCGAAAAAΨAΨAAAGG | |
| | CGGCAGCGGCAGCGGCAGCGGCAGCCGCGGCCCGGΨGA | |
| | ACΨAΨCAΨΨΨΨAGCAACΨAΨAΨGAACCΨGGAΨGGCAG | |
| | CGGCAGCGGCAGCGGCAGCAΨΨGCGAAACGCGΨGCAΨ | |
| | CAGAGCAAAAACCΨGCΨGCGCCGCGCGGGCAGCGGCAG | |
| | CGGCAGCGGCAGCGAΨGAΨAGCΨAΨCGCΨAΨGAΨAΨΨ | |
| | AGCGAAGAAAΨΨGAΨGAΨAAAGGCAGCGGCAGCGGCA | |
| | GCGGCAGCΨΨΨAΨΨAAAAAAAΨΨAACGAAACCΨAΨGG | |
| | CAGCGGCAGCGGCAGCGGCAGCAAACΨGAΨΨCΨGAGC | |
| | GΨGAAAAGCAAAAACCΨGAACAAAGAΨCΨGGGCAGCG | |
| | GCAGCGGCAGCGGCAGCAAAAAACΨGAΨΨCΨGACCAΨ | |
| | ΨAΨΨΨAΨAΨΨCAΨCΨGΨΨΨAΨΨCΨG | |
| 3′-UTR | The 3′ untranslated region comprises two sequence | 2860-3127 |
| | elements derived from the amino-terminal enhancer | |
| | of split (AES) mRNA and the mitochondrial encoded | |
| | 12S ribosomal RNA to confer RNA stability and | |
| | high total protein expression: | |
| | GCΨAGCΨGCCCCΨΨΨCCCGΨCCΨGGGΨACCCCGAGΨCΨ | |
| | CCCCCGACCΨCGGGΨCCCAGGΨAΨGCΨCCCACCΨCCAC | |
| | CΨGCCCCACΨCACCACCΨCΨGCΨAGΨΨCCAGACACCΨC | |
| | CCAAGCACGCAGCAAΨGCAGCΨCAAAACGCΨΨAGCCΨA | |
| | GCCACACCCCCACGGGAAACAGCAGΨGAΨΨAACCΨΨΨA | |
| | GCAAΨAAACGAAAGΨΨΨAACΨAAGCΨAΨACΨAACCCC | |
| | AGGGΨΨGGΨCAAΨΨΨCGΨGCCAGCCACACCCΨGGAGC | |
| | ΨAGC | |
| poly(A) | A 110-nucleotide poly(A)-tail consisting of a | 3128-3237 |
| | stretch of 30 adenosine residues, followed by | |
| | a 10-nucleotide linker sequence and another | |
| | 70 adenosine residues: | |
| | AAAAAAAAAAAAAAAAAAAAAAAAAAAAAAGCAUAUG | |
| | ACUAAAAAAAAAAAAAAAAAAAAAAAAAAAAAAAAAA | |
| | AAAAAAAAAAAAAAAAAAAAAAAAAAAAAAAAAAAA | |

mRNA MANUFACTURING METHOD

The coding mRNA of the invention may be prepared using any method known in the art, including chemical synthesis, such as e.g. solid phase mRNA synthesis, as well as in vitro methods, such as mRNA in vitro transcription reactions.

In a preferred embodiment, the coding mRNA, preferably the mRNA, is obtained by mRNA in vitro transcription.

In preferred embodiments, the cap1 structure of the coding mRNA of the invention is formed using co-transcriptional capping using tri-nucleotide cap analogs m7G(5′)ppp(5′)(2′OMeA)pG or m7G(5′)ppp(5′)(2′OMeG)pG. A preferred cap1 analog that may suitably be used in manufacturing the coding mRNA of the invention is m7G(5′)ppp(5′)(2′OMeA)pG.

In embodiments, the nucleotide mixture used in mRNA in vitro transcription may additionally contain modified nucleotides as defined herein. In that context, preferred modified nucleotides comprise pseudouridine (y), N1-methylpseudouridine, 5-methylcytosine, and 5-methoxyuridine. In particular embodiments, uracil nucleotides in the nucleotide mixture are replaced (either wholly or partially) by pseudouridine and/or N1-methyl pseudouridine to obtain a modified coding mRNA.

In preferred embodiments, the nucleotide mixture (i.e., the fraction of each nucleotide in the mix) used for mRNA in vitro transcription reactions may be optimized for the given mRNA sequence.

In a further preferred embodiment, the coding mRNA, particularly the purified coding mRNA, is lyophilized. The mRNA of the invention, particularly the purified mRNA, may also be dried using spray-drying or spray-freeze drying.

In preferred embodiments, the coding mRNA of the invention is a purified mRNA, particularly purified mRNA that has a higher purity after specific purification steps (e.g., HPLC, TFF, Oligo d (T) purification, precipitation steps) than the starting material (e.g., in vitro transcribed mRNA). Typical impurities that are essentially not present in purified mRNA comprise peptides or proteins (e.g., enzymes derived from DNA-dependent mRNA in vitro transcription, e.g., mRNA polymerases, mRNAses, pyrophosphatase, restriction endonuclease, DNase), spermidine, BSA, abortive mRNA sequences, mRNA fragments (short double-stranded mRNA fragments, abortive sequences, etc.), free nucleotides (modified nucleotides, conventional NTPs, cap analog), template DNA fragments, buffer components (HEPES, TRIS, MgCI2) etc.

Other potential impurities derived from, e.g., fermentation procedures, comprise bacterial impurities (bioburden, bacterial DNA) or impurities derived from purification procedures (organic solvents, etc.).

### Pharmaceutical Composition

A second aspect relates to a composition comprising at least one coding mRNA of the first aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the mRNA of the first aspect). In preferred embodiments of the second aspect, the composition comprises at least one mRNA encoding peptides or proteins of a malaria parasite according to the first aspect, or an immunogenic fragment or immunogenic variant thereof, wherein the composition is to be, preferably, administered intramuscularly or intradermal.

Preferably, intramuscular or intradermal administration of the the composition results in the expression of the encoded antigen in a subject. Preferably, the composition of the second aspect is suitable for a vaccine suitable for malaria.

The composition may comprise a safe and effective amount of the mRNA, as specified herein. As used herein, “safe and effective amount” is an amount of the mRNA sufficient to result in the encoded CSP antigenic protein's expression and/or activity. At the same time, a “safe and effective amount” is small enough to avoid serious side effects.

In the context of the invention, a “composition” refers to any type of composition in which the specified ingredients (e.g., mRNA encoding CSP, e.g., in association with a polymeric carrier or LNP), may be incorporated, optionally along with any further constituents, usually with at least one pharmaceutically acceptable carrier or excipient. The composition may be a dry composition, such as a powder or granules, or a solid unit, such as a lyophilized form. Alternatively, the composition may be liquid, and each constituent may be independently incorporated in dissolved or dispersed (e.g., suspended or emulsified) form.

In a preferred embodiment of the second aspect, the composition comprises mRNA coding at least one protein or peptide and, optionally, at least one pharmaceutically acceptable carrier or excipient.

In particularly preferred embodiments of the second aspect, the composition comprises at least one coding mRNA, wherein the coding mRNA includes or consists of an mRNA sequence that is identical or at least 70% to 99% to a nucleic acid sequence selected from the group consisting of epitopes chosen (Table 1), and, optionally, at least one pharmaceutically acceptable carrier or excipient.

The term “pharmaceutically acceptable carrier” or “pharmaceutically acceptable excipient” as used herein preferably includes the liquid or non-liquid basis of the composition for administration. If the composition is liquid, the carrier may be water, e.g., pyrogen-free water; isotonic saline or buffered (aqueous) solutions, e.g., phosphate, citrate, etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium, and, optionally, potassium salts may occur in the form of their halogenides, e.g., chlorides, iodides, or bromides, in the form of their hydroxides, carbonates, hydrogen carbonates, or sulfates, etc.

Furthermore, organic anions of the cations may be in the buffer. Accordingly, in embodiments, the mRNA composition of the invention may comprise pharmaceutically acceptable carriers or excipients using one or more pharmaceutically acceptable carriers or excipients to, e.g., increase stability, increase cell transfection, permit the sustained or delayed, increase the translation of encoded epitopes. In addition to traditional excipients such as all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers, diluents, or encapsulating compounds, which are suitable for administration to a subject, may also be used.

The term “compatible,” as used herein, is that the constituents of the composition are capable of being mixed with mRNA and, optionally, a plurality of mRNAs of the composition in such a manner that no interaction occurs, which would substantially reduce the biological activity or the pharmaceutical effectiveness of the composition under typical use conditions (e.g., intramuscular, or intradermal administration).

Pharmaceutically acceptable carriers or excipients must have sufficiently high purity and sufficiently low toxicity to make them suitable for administration to a subject to be treated. Compounds that may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such for example, sodium carboxymethylcellulose, ethyl cellulose, cellulose acetate; powdered tragacanth; malt; gelatin; tallow; solid glidants, such as, for example, stearic acid, magnesium stearate; calcium sulfate; vegetable oils, such as, for instance, groundnut oil, cottonseed oil, sesame oil, olive oil, corn oil and oil from Theobroma; polyols, such as, for example, polypropylene glycol, glycerol, sorbitol, mannitol and polyethylene glycol; alginic acid.

At least one pharmaceutically acceptable carrier or excipient of the composition may preferably be selected to be suitable for intramuscular or intradermal delivery of the the composition. Accordingly, the composition is preferably a pharmaceutical composition suitable for intramuscular or intradermal administration.

The pharmaceutical composition is contemplated for use, but is not limited to, humans and other primates; mammals, including commercially relevant mammals such as cattle, pigs, horses, sheep, cats, dogs, mice, and rats; and birds, including commercially relevant birds such as poultry, chickens, ducks, geese, and turkeys.

Pharmaceutical compositions of the present invention may suitably be sterile and pyrogen-free. Furthermore, one or more compatible solid or liquid filler diluents or encapsulating compounds, which are suitable for administration to a person, may also be used. The term “compatible,” as used herein, is that the constituents of the composition are capable of being mixed with mRNA and, optionally, the further coding mRNA of the composition in such a manner that no interaction occurs, which would substantially reduce the biological activity or the pharmaceutical effectiveness of the composition under typical use conditions.

In embodiments, the composition as defined herein may comprise a plurality or at least more than one of the coding mRNA species as defined in the context of the first aspect of the invention.

In embodiments, mRNA comprised in the composition is a bi- or multicistronic nucleic acid, particularly a bi- or multicistronic nucleic acid as defined herein, which encodes at least two, three, four, five, six, seven, eight, nine, ten, eleven or twelve distinct antigenic peptides or protein derived from the same malaria parasite and/or a malaria parasite.

In embodiments, the composition as defined herein may comprise a plurality or at least more than one of the coding mRNA species as defined in the context of the first aspect of the invention.

In an embodiment, the composition may comprise 2-10 or even more different peptides or protein-coding mRNA as defined in the context of the first aspect, each encoding at least one antigenic peptide or protein derived from genetically the same malaria parasite or a fragment or variant thereof. Particularly, the (genetically) same malaria parasite expresses (essentially) the same repertoire of proteins or peptides, wherein all proteins or peptides have (essentially) the same amino acid sequence. Particularly, the (genetically) same malaria parasite expresses essentially the same proteins, peptides, or polyproteins, wherein these proteins, peptides, or polyproteins preferably do not differ in their amino acid sequence(s).

In other embodiments, the composition comprises at least one or more epitopes derived from species encoding a different malaria antigen selected from AMA1, CSP, CyRPA, EMP1, LSA, LSA1, MSP1, Pfs23, Pfs230, pfs25, Pfs28, Pfs45, Pfs48, RH5, RIPR, SSP2, TRAP, Vagr2CSA, and VAR2CSA, or a combination thereof.

### Complexation

In a preferred embodiment of the second aspect, the mRNA is complexed or associated with to obtain a formulated composition. A formulation in that context may have the function of a transfection agent. A formulation in that context may also have the function of protecting the coding mRNA from degradation.

In a preferred embodiment of the second aspect, the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

The term “cationic or polycationic compound” as used herein will be recognized and understood by the person of ordinary skill in the art and is, for example, intended to refer to a charged molecule, which is positively charged at a pH value ranging from about 1 to 9. Accordingly, a cationic component, e.g., a cationic peptide, cationic protein, cationic polymer, cationic polysaccharide, or cationic lipid, may be any positively charged compound or polymer that is positively charged under physiological conditions. A “cationic or polycationic peptide or protein” may contain at least one positively charged amino acid or more than one positively charged amino acid, e.g., selected from Arg, His, Lys, Glu, Asp, or Orn. Accordingly, “polycationic” components are also within the scope of exhibiting more than one positive charge under the given conditions.

Cationic or polycationic compounds, being particularly preferred in this context, may be selected from the following list of cationic or polycationic peptides or proteins of fragments thereof. protamine, nucleoline, spermine or spermidine, or other cationic peptides or proteins, such as poly-L-lysine (PLL), poly-arginine, essential polypeptides, cell-penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, or histones. The nucleic acid, as defined herein, preferably the mRNA as defined herein, is more complex with one or more polycations, preferably with protamine or oligofectamine, most preferably with protamine.

Coding mRNA is complexed with protamine in a preferred embodiment of the second aspect.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethylene glycol); etc.

In this context, it is particularly preferred that coding mRNA is complex or at least partially complexed with a cationic or polycationic compound and/or a polymeric carrier, preferably cationic proteins or peptides.

In a preferred embodiment of the second aspect, the composition comprises at least one coding mRNA complexed with one or more cationic or polycationic compounds, preferably protamine, and at least one free coding mRNA. In this context, at least one coding mRNA is particularly preferred to be complexed or at least partially complexed with protamine. Preferably, the molar ratio of the nucleic acid, particularly the mRNA of the protamine-complexed mRNA to the free mRNA, may be selected from a molar ratio of about 0.001:1 to about 1:0.001, including a ratio of about 1:1. Suitably, the complexed mRNA is complexed with protamine by addition of protamine-trehalose solution to the mRNA sample at a mRNA: protamine weight to weight ratio (w/w) of 2:1.

In a preferred embodiment of the second aspect, coding mRNA is complexed or partially complexed, with at least one cationic or polycationic protein, peptide, or a combination thereof.

According to embodiments, the composition of the present invention comprises the coding mRNA as defined in the context of the first aspect and a polymeric carrier.

The term “polymeric carrier” as used herein will be recognized and understood by the person of ordinary skill in the art and is, e.g., intended to refer to a compound that facilitates transport and complexation of another compound (e.g., cargo mRNA). A polymeric carrier is typically a carrier that is formed of a polymer. A polymeric carrier may be associated with its cargo (e.g., coding mRNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3, 3′-dithiobispropionimidate (DTBP), polyethylene imine biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly-aminoester, poly(4-hydroxy-L-proine ester) (PUP), poly(allylamine), poly(a-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE EA), galactosylated chitosan, N-dodecylated chitosan, histone, collagen and dextran-spermine. In one embodiment, the polymer may be an inert polymer such as, but not limited to, PEG. In one embodiment, the polymer may be cationic, such as, but not limited to, PEI, PLL, TETA, poly(allylamine), Poly(N-ethyl-4-vinylpyridinium bromide), pHPMA, and pDMAEMA. In one embodiment, the polymer may be a biodegradable PEI such as, but not limited to, DSP, DTBP, and PEIC. In one embodiment, the polymer may be biodegradable, such as, but not limited to, histidine-modified PLL, SS-PAEI, poly\hat{ }-aminoester), PUP, PAGA, PLGA, PPZ, PPE, PPA and PPE-EA.

When PEI is present, it may be branched PEI of a molecular weight ranging from 10 to 40 kDa, e.g., 25 kDa branched PEI (Sigma #408727). In some embodiments, the polymer-based nanoparticle comprises PEI. In some embodiments, the PEI is branched PEI. PEI may be a branched PEI of a molecular weight ranging from 10 to 40 kDa, e.g., 25 kDa. In some embodiments, the PEI is linear. In some embodiments, the nanoparticle has a size of or less than about 60 nm (e.g., of or less than about 55 nm, of or less than about 50 nm, of or less than about 45 nm, of or less than about 40 nm, of or less than about 35 nm, of or less than about 30 nm, or of or less than about 25 nm). Suitable nanoparticles may range from 25 to 60 nm, e.g., 30 to 50 nm.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used in the present invention may comprise mixtures of cationic peptides, proteins, polymers, and optionally further components as defined herein, which are crosslinked by disulfide bonds (via —SH groups).

In a particularly preferred embodiment, the polymeric carrier is a peptide polymer, preferably a polyethylene glycol/peptide polymer as defined above, and a lipid component, preferably a lipidoid component. In a preferred embodiment of the second aspect, coding mRNA of the first aspect is complexed or associated with a polymeric carrier, preferably with a polyethylene glycol/peptide polymer as defined above, and a lipidoid component, wherein the lipidoid component is a compound.

According to preferred embodiments, the peptide polymer comprises preferably lipidoid 3-C12-OH, which is used to complex coding mRNA of the first aspect to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid.

### Encapsulation/Complexation in LNPS:

In preferred embodiments of the second aspect, coding mRNA is complexed, encapsulated, partially encapsulated, or associated with one or more lipids (e.g., cationic lipids and/or neutral lipids), thereby forming liposomes, lipid nanoparticles (LNPs), lipoplexes, and nanoliposomes.

The liposomes, lipid nanoparticles (LNPs), lipoplexes, and nanoliposomes—incorporated mRNA may be entirely or partially located in the interior space of the liposomes, lipid nanoparticles (LNPs), lipoplexes, and nanoliposomes, within the membrane, or associated with the exterior surface of the membrane. Incorporating nucleic acid into liposomes is also referred to herein as “encapsulation” wherein the mRNA is entirely contained within the interior space of the liposomes, lipid nanoparticles (LNPs), lipoplexes, and nanoliposomes. The purpose of incorporating an mRNA into liposomes, lipid nanoparticles (LNPs), lipoplexes, and nanoliposomes is to protect the mRNA from an environment that may contain enzymes or chemicals that degrade mRNA and systems or receptors that cause the rapid excretion of the mRNA. Moreover, incorporating an mRNA into liposomes, lipid nanoparticles (LNPs), lipoplexes, and nanoliposomes may promote the uptake of the mRNA and, hence, enhance the therapeutic effect of the mRNA encoding antigenic peptides. Accordingly, incorporating an mRNA into liposomes, lipid nanoparticles (LNPs), lipoplexes, and nanoliposomes may be particularly suitable for a vaccine, e.g., intramuscular or intradermal administration.

In this context, “complexed” or “associated” refers to the essentially stable combination of coding mRNA of the first aspect with one or more lipids into larger complexes or assemblies without covalent binding.

The term “lipid nanoparticle,” also referred to as “LNP,” is not restricted to any particular morphology and includes any morphology generated when a cationic lipid and optionally one or more further lipids are combined, e.g., in an aqueous environment and the presence of mRNA. For example, a liposome, a lipid complex, a lipoplex, and the like are within the scope of a lipid nanoparticle (LNP).

Liposomes, lipid nanoparticles (LNPs), lipoplexes, and nanoliposomes can be of different sizes, such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin, that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the mRNA of the first aspect to a target tissue.

Accordingly, in preferred embodiments of the second aspect, mRNA is complexed with one or more lipids, forming lipid nanoparticles (LNP), wherein the LNP is particularly suitable for intramuscular and intradermal administration.

LNPs typically comprise a cationic lipid and one or more excipients selected from neutral lipids, charged lipids, steroids, and polymer-conjugated lipids (e.g., PEGylated lipids). The coding mRNA may be encapsulated in the lipid portion of the LNP or an aqueous space enveloped by some or the entire lipid portion of the LNP. The coding mRNA or a portion thereof may also be associated and complexed with the LNP. An LNP may comprise any lipid capable of forming a particle to which the nucleic acids are attached or in which one or more nucleic acids are encapsulated. Preferably, the LNP comprising nucleic acids comprises one or more cationic lipids and stabilizing lipids. Stabilizing lipids include neutral lipids and PEGylated lipids.

The cationic lipid of an LNP may be cationisable, i.e., it becomes protonated as the pH is lowered below the pK of the ionizable group of the lipid but is progressively more neutral at higher pH values. At pH values below the pK, the lipid can be associated with negatively charged nucleic acids. In certain embodiments, the cationic lipid comprises a zwitterionic lipid that assumes a positive charge on pH decrease.

The LNP may comprise any further cationic or cationisable lipid, i.e., any of several lipid species that carry a net positive charge at a selective pH, such as physiological pH.

Such lipids include, but are not limited to, DSDMA, N, N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt, N-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-Dilinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (g-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-DLin-K-DMA) or analogs thereof, (3aR,5s,6aS)—N,N-dimethyl-2,2-di((9Z, 12Z)-octadeca-9, 12-dienyl)tetrahydro-3aH-cyclopenta[d][1,3]dioxol-5-amine, (6Z,9Z,28Z,31 Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d][1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DL in-K-DMA;XTC), NC98-5 (4,7, 13-tris(3-oxo-3-(undecylamino)propyl)-NI,N 16-diundecyl-4,7, 10,13-tetraazahexadecane-1, 16-diamide), (6Z,9Z,28Z,31Z)-heptatriaconta-6, 9, 28, 31-tetraen-19-yl 4-(dimethylamino) butanoate (DLin-M-C3-DMA), 3-((6Z,9Z,28Z,31 Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing.

In some embodiments, the lipid is selected from the 98N12-5, C12-200, and ckk-E12 groups.

In one embodiment, the further cationic lipid is an amino lipid including but not limited to 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N, N-dioleylamino)-1,2-propanediol (DOAP), 1,2-dilinoleyloxo-3-(2-N, N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

### Adjuvants:

According to further embodiments, the composition of the second aspect may comprise at least one adjuvant. Suitably, the adjuvant is preferably added to enhance the immunostimulatory properties of the composition.

The term “adjuvant” as used herein will be recognized and understood by the person of ordinary skill in the art and is, for example, intended to refer to a pharmacological and immunological agent that may modify, e.g., enhance, the effect of other agents (herein: the impact of the coding mRNA), or that may be suitable to support administration and delivery of the composition. The term “adjuvant” refers to a broad spectrum of substances. Typically, these substances can increase the immunogenicity of antigens. For example, adjuvants may be recognized by the innate immune systems and, e.g., may elicit an innate immune response (a nonspecific immune response). “Adjuvants” typically do not elicit an adaptive immune response.

In the context of the invention, adjuvants may enhance the effect of the antigenic peptide or protein provided by the coding RNA. In that context, at least one adjuvant may be selected from any adjuvant known to a skilled person and suitable for the present case, i.e., supporting the induction of an immune response in a subject, e.g., a human subject.

The composition of the second aspect may comprise, besides the components specified herein, at least one further component which may be selected from the group consisting of further antigens (e.g., in the form of a peptide or protein) or further antigen-encoding nucleic acids, a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and/or an adjuvant nucleic acid, preferably an immunostimulatory RNA (isRNA), e.g., CpG-RNA etc.

The LNP formulation is also an adjuvant.

### Vaccine:

In a third aspect, the present invention provides a malaria vaccine wherein the vaccine comprises the coding RNA of the first aspect and, optionally, the composition of the second aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the RNA of the first aspect).

The term “vaccine” will be recognized and understood by the person of ordinary skill in the art and is, for example, intended to be a prophylactic or therapeutic material providing at least one epitope or antigen, preferably an immunogen. In the context of the invention, the antigen or antigenic function is provided by the inventive coding RNA of the first aspect (the RNA comprising a coding sequence encoding an antigenic peptide or protein derived from CSP) or the composition of the second aspect (comprising the RNA of the first aspect).

In preferred embodiments of the third aspect, the vaccine comprising the first aspect's mRNA or the second aspect's composition elicits an adaptive immune response, preferably an adaptive immune response against a malaria parasite.

In preferred embodiments of the third aspect, the vaccine comprising the first aspect's RNA or the second aspect's composition induces strong humoral and cellular immune responses, both B-cell and preferably strong CD4+ and CD8+ T-cell responses.

According to a preferred embodiment of the third aspect, the vaccine, as defined herein, may further comprise a pharmaceutically acceptable carrier and optionally at least one adjuvant as specified in the context of the second aspect.

In some embodiments, the “safe and effective amount” is a dose equivalent to at least 2-fold, at least 4-fold, at least 10-fold, at least 100-fold, and at least 1000-fold reduction in the standard of care dose of a recombinant malaria protein vaccine, wherein the anti-antigenic polypeptide antibody titer produced in the subject is equivalent to an anti-antigenic polypeptide antibody titer produced in a control subject administered the standard of care dose of a recombinant malaria protein vaccine, a purified malaria protein vaccine, a live attenuated malaria vaccine, an inactivated malaria vaccine or a malaria VLP vaccine. In some embodiments, the control is an anti-antigenic polypeptide antibody titer produced in a subject who has been administered a virus-like particle (VLP) vaccine comprising structural proteins of malaria.

The vaccine can be used according to the invention for human medical and veterinary medical purposes (mammals, vertebrates, avian species), as a pharmaceutical composition, or as a vaccine.

### Medical Use:

Further, the present invention relates to the first medical use of the coding RNA of the first aspect, the composition of the second aspect, and the vaccine of the third aspect.

Accordingly, the RNA of the first aspect, the composition of the second aspect, and the vaccine of the third aspect are used as a medicament.

The present invention provides several applications and uses of the coding RNA of the first aspect, the composition of the second aspect, or the vaccine of the third aspect.

In particular, RNA, composition, and vaccine may be used for human medical and veterinary medical purposes, preferably for human medical purposes.

Said RNA composition is used as a medicament for human medical purposes. The RNA, composition, and vaccine may suit young infants, newborns, immunocompromised recipients, pregnant and breast-feeding women, and older adults.

In yet another aspect, the present invention relates to the second medical use of the coding RNA of the first aspect, the composition of the second aspect, and the vaccine of the third aspect.

In embodiments, the RNA of the first aspect, the composition of the second aspect, and the vaccine of the third aspect are for use in the treatment or prophylaxis of an infection with a pathogen (e.g., a protozoan parasite) with a malaria parasite, or a disorder related to such an infection.

In embodiments, the RNA of the first aspect, the composition of the second aspect, the vaccine of the third aspect, is for use in the treatment or prophylaxis of an infection with a malaria parasite, in particular with Plasmodium falciparum (Pf), Plasmodium knowlesi (Pk), Plasmodium ovale (Po), Plasmodium simiovale (Ps), and Plasmodium vivax (Pv), Plasmodium malariae (Pm), Plasmodium ovale curtisi (Poc), Plasmodium ovale wallikeri (Pow), or Plasmodium berghei (Pb).

In preferred embodiments, the RNA of the first aspect, the composition of the second aspect, and the vaccine of the third aspect are used in the treatment or prophylaxis of infection with Plasmodium falciparum (Pf). The mRNA of the first aspect, the composition of the second aspect, and the vaccine of the third aspect may be used in the treatment or prophylaxis of an infection with a malaria parasite, in particular with Plasmodium falciparum (Pf), Plasmodium knowlesi (Pk), Plasmodium ovale (Po), Plasmodium simiovale (Ps), and Plasmodium vivax (Pv), Plasmodium malariae (Pm), Plasmodium ovale curtisi (Poc), Plasmodium ovale wallikeri (Pow), or Plasmodium berghei (Pb), or a disorder related to such an infection, for human and also for veterinary medical purposes, preferably for human medical purposes.

As used herein, “a disorder related to a malaria infection” may preferably comprise a typical symptom or a complication of a malaria infection.

Particularly, the coding RNA of the first aspect, the composition of the second aspect, and the vaccine of the third aspect, may be used in a method of prophylactic (pre-exposure prophylaxis or post-exposure prophylaxis) and/or treatment of infections caused by a malaria parasite.

The composition or the vaccine as defined herein may preferably be administered locally. An intradermal, subcutaneous, intranasal, or intramuscular route may administer composition or vaccines. Inventive compositions or vaccines of the invention are, therefore, preferably formulated in liquid (or sometimes in solid) form. In embodiments, conventional needle or needle-free jet injection may administer the inventive vaccine. Preferred in that context is the RNA, the composition, and the vaccine, which is administered by intramuscular needle injection.

The term jet injection”, as used herein, refers to a needle-free injection method wherein a fluid (vaccine, composition of the invention) containing, e.g., at least one RNA of the first aspect is forced through an orifice, thus generating an ultra-fine liquid stream of high pressure that is capable of penetrating mammalian skin and depending on the injection settings, subcutaneous tissue or muscle tissue. In principle, the liquid stream perforates the skin, through which the liquid stream is pushed into the target tissue. Jet injection is preferred for intradermal, subcutaneous, or intramuscular injection of the RNA, the compositions, and the vaccines disclosed herein.

In embodiments, the RNA as comprised in a composition or vaccine as defined herein is provided from about 100 ng to about 500 ug.

In some embodiments, a vaccine comprising the coding RNA is formulated effectively to produce an antigen-specific immune response in a subject. In some embodiments, the effective dose is 1 ug to 100 ug.

In some embodiments, the subject is about 5 years old or younger or 70 years old or older.

1. A coding mRNA for a vaccine comprising
a) at least one 5′-cap structure
b) at least one heterologous 5′ untranslated region (5′-UTR)
c) at least one heterologous 3′ untranslated region (3′-UTR)
d) at least one signal peptide;
e) at least one poly(A) tail
e) at least one coding sequence for at least one antigenic protein of a malaria parasite linked operably to the 3′-UTR and the 5′-UTR.

2. The coding mRNA of claim 1, wherein the coding sequence contains at least one malaria parasite epitope.

3. The coding mRNA of claim 2, wherein multiple epitopes of malaria parasite are linked in a single protein sequence.

4. The coding mRNA of claim 1, wherein the 5′-cap comprises m7,3′-GpppG, m7G(5′)ppp(5′)(m7G), m7GpppN, m7GpppNm, m7GpppNmNm, m7GpppNmNmN, m7GpppN, m7G+m3′-5′-ppp-5′-Am, or m7GpppG.

5. The coding mRNA of claim 1, wherein the mRNA comprises a poly(A) sequence, preferably comprising 30 to 150 adenosine nucleotides.

6. The coding mRNA of claim 2, where the cap is m7G+m3′-5′-ppp-5′-Am.

7. The coding mRNA of claim 1, wherein the heterologous signal peptide is selected from a group of signal peptides comprising
TTCGTGTTCCTGGTGCTGCTGCCTCTGGTGTCCAGCCAGTGTGTG,
ATGTTCGTGTTCCTGGTGCTGCTGCCTCTGGTGTCCAGCCAGTGTGTG,
MAKNLILALILAFVLTATVA,
MALLWSVLLGLLQLGAVAPR,
MKFLVNVALVFMVVYISYIYA,
MKKNLILAVLALVSSILAN,
MKKQSTIALALLPLLFTPVTKA,
MKKTAIAIAVALAGFATVAQA,
MKKTSFFLILVLLFTGCSG,
MKLLTALALVVTMVASV,
MKNWILVVLAVLAAVSSLAHA,
MKWVTFISLLFLFSSAYS,
MKYLLPTAAAGLLLLAAQPAMA,
MLLSVPLLLGLLGLAVA,
and
MRRLLLTGAAALGCTAA.

8. The coding mRNA of claim 1, wherein the malaria parasite comprises Plasmodium falciparum (Pf), Plasmodium falciparum 3D7, Plasmodium knowlesi (Pk), Plasmodium ovale (Po), Plasmodium simiovale (Ps), Plasmodium vivax (Pv), and Toxoplasma gondii or a combination thereof.

9. The coding mRNA of claim 1, wherein the antigens of malaria pathogen comprises (1) Circumsporozoite protein (CS); Q5R2L8 Q5R2L8_PLAFA: Plasmodium falciparum, (2) Merozoite Surface Protein 1 (MSP1): P50495 MSP1_PLAFP (Uganda); Plasmodium falciparum (isolate Palo Alto/Uganda), (3) Apical Membrane Antigen 1 (AMA1): P22621⋅AMA1_PLAFF New Guinea: Plasmodium falciparum (isolate FC27/Papua New Guinea), (4) Erythrocyte Binding Antigens (EBAs) A0A023ZZ81 1_PLAFA: Plasmodium falciparum Vietnam Oak-Knoll (FVO) (no rank), (5) Rhoptry Neck Proteins (RONs): B6KV60⋅RON2_TOXGM: Reticulocyte-binding protein homolog 5: Toxoplasma gondii (strain ATCC 50611/Me49), (6) Rhoptry neck protein 2: I0J1F2 I0J1F2_PLAVI: Plasmodium vivax, (7) Rhoptry neck protein 2: I0J1I4 I0J1I4_PLAVI: Plasmodium vivax, and (8) PfRh Proteins: Q8IFM5 RH5_PLAF7: Plasmodium falciparum Vietnam Oak-Knoll (FVO) (no rank), of a combination thereof.

10. The coding mRNA of claim 1, wherein the epitope combines several epitopes derived from the same or different species of malaria pathogen and linked together by a linker.

11. The coding mRNA of claim 2, wherein at least one open reading frame is independently linked operably to the 3′-UTR and 5′-UTR encoding a single antigenic protein or multiple of a selected malaria parasite, administered separately, or combined in a single formulation and administered as a single injection.

12. The coding mRNA of claim 3, wherein the linker comprises AAAGY (Alanine-Alanine-Alanine-Glycine-Tyrosine), AAY (alanine and tyrosine), APAAP (Alanine-Proline-Alanine-Alanine-Proline), EAAAK (Glutamic Acid-Alanine Linker), EFGGG (Glutamic Acid-Phenylalanine-Glycine-Glycine-Glycine), GGAGG (A slight variation of the GGGGS linker with an alanine residue), GGGGS (Glycine-Serine Linker), GGGS (linker is one of the simplest and commonly used linkers), GGGSGGG (linker consists of a longer sequence of glycine (G) and serine (S) residues), GGGSGGGGSGGG (linker with multiple glycine and serine residues), GGPGG (Glycine-Glycine-Proline-Glycine-Glycine), GGSGG: (An inversion of the standard GGGGS linker), GGSSG (Glycine-Glycine-Serine-Serine-Glycine), GGTGG (Glycine-Glycine-Threonine-Glycine-Glycine), GPGP (Glycine-Proline-Glycine-Proline), GPGPG (Glycine-Proline-Glycine-Proline-Glycine), GPGS (Glycine-Proline-Glycine-Serine), GSGPG (Glycine-Serine-Glycine-Proline-Glycine), GSSG (Glycine-Serine-Serine-Glycine), GSSGG (Glycine-Serine-Serine-Glycine-Glycine), GSTSG (Glycine-Serine-Threonine Linker), KK (Lysine-Lysine), KKKGS (Lysine-Glycine-Serine Linker), KLPGWSG (A specific sequence), LEGGGS (Leucine-Glutamic Acid-Glycine-Glycine-Serine), NPGP (Asparagine-Proline-Glycine-Proline), SGGGG (A variant of the GGGGS linker), SGSGS (Serine-Glycine-Serine-Glycine-Serine), SSGGG (Serine-Serine-Glycine-Glycine-Glycine), SSGSS (Serine-Serine-Glycine-Serine-Serine), TGGGS (Threonine-Glycine-Glycine-Glycine-Serine), TPGTG (Threonine-Proline-Glycine-Threonine-Glycine), TPP (Proline-Proline-Threonine), TPTPPT (Threonine-Proline-Threonine-Proline-Proline-Threonine), TSGSG: (A variant of the GSTSG linker), TSGTSG (Threonine-Serine-Glycine-Threonine-Serine-Glycine), and XTEN (A synthetic, non-immunogenic linker), or a combination thereof.

13. The coding mRNA of claim 3, wherein the target protein and peptide sequences are linked using the linker GSGSGSGS to provide an antigen comprises
MMRKLAILSVSSFLFVEALFQEYQCYGSSSNTRVL
NELNYDNAGTNLYNELEMNYYGKQENWYSLKKNSR
SLGENDDGNNNNGDNGREGKDEDKRDGNNEDNEKL
RKPKHKKLKQPGDGNPDPNANPNVDPNANPNVDPN
ANPNVDPNANPNANPNANPNANPNANPNANPNANP
NANPNANPNANPNANPNANPNANPNANPNANPNVD
PNANPNANPNANPNANPNANPNANPNANPNANPNA
NPNANPNANPNANPNANPNANPNANPNANPNANPN
ANPNANPNKNNQGNGQGHNMPNDPNRNVDENANAN
NAVKNNNNEEPSDKHIEQYLKKIKNSISTEWSPCS
VTCGNGIQVRIKPGSANKPKDELDYENDIEKKICK
MEKCSSVFNVVNSSIGLIMVLSFLFLNGSGSGSGS
VTHESYQELVKKLEALEDAVGSGSGSGSGLFHKEK
MILNEEEITTKGAGSGSGSGSQEGISYYEKVLAKY
GSGSGSGSFLTNIETLYNNLVNKGSGSGSGSGISF
LLILMLILYSFGSGSGSGSLMSPMTLDEMRHFYKD
NKGSGSGSGSSASDQPKQYEQHLTDYGSGSGSGSI
IIASSAAVGSGSGSGSGFKNKNASMIKSAFLGSGS
GSGSVWECKNPYILSTKDVCVPPGSGSGSGSDKNL
LMIKEHILAIAGSGSGSGSDIIGGTDYWGSGSGSG
SGNIDRIYDKNLLMIKGSGSGSGSKYTTPVFPMSA
PGSGSGSGSPSGIRVYATPPAPRPGSGSGSGSYGR
TRNKRYMHRNPGEKYKGGSGSGSGSRGPVNYHFSN
YMNLDGSGSGSGSIAKRVHQSKNLLRRAGSGSGSG
SDDSYRYDISEEIDDKGSGSGSGSFIKKINETYGS
GSGSGSKLILSVKSKNLNKDLGSGSGSGSKKLILT
IIYIHLFIL.

14. The coding mRNA of claim 13, wherein the open reading frame sequence is
AΨGAΨGCGCAAACΨGGCGAΨΨCΨGAGCGΨGAGCAGCΨΨΨCΨGΨΨΨ
GΨGGAAGCGCΨGΨΨΨCAGGAAΨAΨCAGΨGCΨAΨGGCAGCAGCAGC
AACACCCGCGΨGCΨGAACGAACΨGAACΨAΨGAΨAACGCGGGCACC
AACCΨGΨAΨAACGAACΨGGAAAΨGAACΨAΨΨAΨGGCAAACAGGAA
AACΨGGΨAΨAGCCΨGAAAAAAAACAGCCGCAGCCΨGGGCGAAAAC
GAΨGAΨGGCAACAACAACAACGGCGAΨAACGGCCGCGAAGGCAAA
GAΨGAAGAΨAAACGCGAΨGGCAACAACGAAGAΨAACGAAAAACΨG
CGCAAACCGAAACAΨAAAAAACΨGAAACAGCCGGGCGAΨGGCAAC
CCGGAΨCCGAACGCGAACCCGAACGΨGGAΨCCGAACGCGAACCCG
AACGΨGGAΨCCGAACGCGAACCCGAACGΨGGAΨCCGAACGCGAAC
CCGAACGCGAACCCGAACGCGAACCCGAACGCGAACCCGAACGCG
AACCCGAACGCGAACCCGAACGCGAACCCGAACGCGAACCCGAAC
GCGAACCCGAACGCGAACCCGAACGCGAACCCGAACGCGAACCCG
AACGCGAACCCGAACGCGAACCCGAACGCGAACCCGAACGΨGGAΨ
CCGAACGCGAACCCGAACGCGAACCCGAACGCGAACCCGAACGCG
AACCCGAACGCGAACCCGAACGCGAACCCGAACGCGAACCCGAAC
GCGAACCCGAACGCGAACCCGAACGCGAACCCGAACGCGAACCCG
AACGCGAACCCGAACGCGAACCCGAACGCGAACCCGAACGCGAAC
CCGAACGCGAACCCGAACGCGAACCCGAACGCGAACCCGAACGCG
AACCCGAACAAAAACAACCAGGGCAACGGCCAGGGCCAΨAACAΨG
CCGAACGAΨCCGAACCGCAACGΨGGAΨGAAAACGCGAACGCGAAC
AACGCGGΨGAAAAACAACAACAACGAAGAACCGAGCGAΨAAACAΨ
AΨΨGAACAGΨAΨCΨGAAAAAAAΨΨAAAAACAGCAΨΨAGCACCGAA
ΨGGAGCCCGΨGCAGCGΨGACCΨGCGGCAACGGCAΨΨCAGGΨGCGC
AΨΨAAACCGGGCAGCGCGAACAAACCGAAAGAΨGAACΨGGAΨΨAΨ
GAAAACGAΨAΨΨGAAAAAAAAAΨΨΨGCAAAAΨGGAAAAAΨGCAGC
AGCGΨGΨΨΨAACGΨGGΨGAACAGCAGCAΨΨGGCCΨGAΨΨAΨGGΨG
CΨGAGCΨΨΨCΨGΨΨΨCΨGAACGGCAGCGGCAGCGGCAGCGΨGACC
CAΨGAAAGCΨAΨCAGGAACΨGGΨGAAAAAACΨGGAAGCGCΨGGAA
GAΨGCGGΨGGGCAGCGGCAGCGGCAGCGGCAGCGGCCΨGΨΨΨCAΨ
AAAGAAAAAAΨGAΨΨCΨGAACGAAGAAGAAAΨΨACCACCAAAGGC
GCGGGCAGCGGCAGCGGCAGCGGCAGCCAGGAAGGCAΨΨAGCΨAΨ
ΨAΨGAAAAAGΨGCΨGGCGAAAΨAΨGGCAGCGGCAGCGGCAGCGGC
AGCΨΨΨCΨGACCAACAΨΨGAAACCCΨGΨAΨAACAACCΨGGΨGAAC
AAAGGCAGCGGCAGCGGCAGCGGCAGCGGCAΨΨAGCΨΨΨCΨGCΨG
AΨΨCΨGAΨGCΨGAΨΨCΨGΨAΨAGCΨΨΨGGCAGCGGCAGCGGCAGC
GGCAGCCΨGAΨGAGCCCGAΨGACCCΨGGAΨGAAAΨGCGCCAΨΨΨΨ
ΨAΨAAAGAΨAACAAAGGCAGCGGCAGCGGCAGCGGCAGCAGCGCG
AGCGAΨCAGCCGAAACAΨGAΨGAACAGCAΨCΨGACCGAΨΨAΨGGC
AGCGGCAGCGGCAGCGGCAGCAΨΨAΨΨAΨΨGCGAGCAGCGCGGCG
GΨGGGCAGCGGCAGCGGCAGCGGCAGCGGCΨΨΨAAAAACAAAAAC
GCGAGCAΨGAΨΨAAAAGCGCGΨΨΨCΨGGGCAGCGGCAGCGGCAGC
GGCAGCGΨGΨGGGAAΨGCAAAAACCCGΨAΨAΨΨCΨGAGCACCAAA
GAΨGΨGΨGCGΨGCCGCCGGGCAGCGGCAGCGGCAGCGGCAGCGAΨ
AAAAACCΨGCΨGAΨGAΨΨAAAGAACAΨAΨΨCΨGGCGAΨΨGCGGGC
AGCGGCAGCGGCAGCGGCAGCGAΨAΨΨAΨΨGGCGGCACCGAΨΨAΨ
ΨGGGGCAGCGGCAGCGGCAGCGGCAGCGGCAACAΨΨGAΨCGCAΨΨ
ΨAΨGAΨAAAAACCΨGCΨGAΨGAΨΨAAAGGCAGCGGCAGCGGCAGC
GGCAGCAAAΨAΨACCACCCCGGΨGΨΨΨCCGAΨGAGCGCGCCGGGC
AGCGGCAGCGGCAGCGGCAGCCCGAGCGGCAΨΨCGCGΨGΨAΨGCG
ACCCCGCCGGCGCCGCGCCCGGGCAGCGGCAGCGGCAGCGGCAGC
ΨAΨGGCCGCACCCGCAACAAACGCΨAΨAΨGCAΨCGCAACCCGGGC
GAAAAAΨAΨAAAGGCGGCAGCGGCAGCGGCAGCGGCAGCCGCGGC
CCGGΨGAACΨAΨCAΨΨΨΨAGCAACΨAΨAΨGAACCΨGGAΨGGCAGC
GGCAGCGGCAGCGGCAGCAΨΨGCGAAACGCGΨGCAΨCAGAGCAAA
AACCΨGCΨGCGCCGCGCGGGCAGCGGCAGCGGCAGCGGCAGCGAΨ
GAΨAGCΨAΨCGCΨAΨGAΨAΨΨAGCGAAGAAAΨΨGAΨGAΨAAAGGC
AGCGGCAGCGGCAGCGGCAGCΨΨΨAΨΨAAAAAAAΨΨAACGAAACC
ΨAΨGGCAGCGGCAGCGGCAGCGGCAGCAAACΨGAΨΨCΨGAGCGΨG
AAAAGCAAAAACCΨGAACAAAGAΨCΨGGGCAGCGGCAGCGGCAGC
GGCAGCAAAAAACΨGAΨΨCΨGACCAΨΨAΨΨΨAΨAΨΨCAΨCΨGΨΨΨ
AΨΨCΨG.

15. The coding mRNA of claims 1 and 2 of any preceding claims, wherein the coding mRNA is a self-replicating RNA, a circular RNA, or a replicon RNA.

16. The coding mRNA of claim 1, wherein the composition optionally comprises at least one pharmaceutically acceptable carrier.

17. The coding mRNA of claim 3, wherein the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

18. The coding mRNA of claim 17, wherein the coding mRNA is complexed or associated with one or more lipids, thereby forming liposomes, lipid nanoparticles, lipoplexes, and/or nanoliposomes.

19. The coding mRNA of claim 18, wherein at least one coding mRNA is complexed with one or more lipids, forming lipid nanoparticles (LNP).

20. The coding mRNA of claim 19, wherein the LNP essentially consists of
(i) at least one cationic lipid;
(ii) at least one neutral lipid;
(iii) at least one steroid or steroid analog; and
(iv) at least once a PEG-lipid,
Wherein (i) to (iv) are in a molar ratio of about 20-60% cationic lipid, 5-25% neutral lipid, 25-55% sterol, and 0.5-15% PEG-lipid.

21. The coding mRNA of claims 1 and 2, wherein the vaccine elicits an adaptive immune response.

22. A method of treating or preventing a disorder, wherein the method comprises applying or administering to a subject in need thereof the coding mRNA as defined in any one of the above claims, the composition as defined in any one of the claims, or the vaccine as defined in any one of claims.

23. The method of claim 22, wherein the disorder is an infection with malaria or related to such an infection.

24. The method of claim 23, wherein the subject in need is mammalian, preferably a human subject.


SEQUENCE LISTING (42 sequences)

SEQ ID NO: 1 - RNA, 42 nt, synthetic construct
  source 1..42: mol_type = other RNA
gaaaaacagaccggccccacagaccagagagaacccgccacc

SEQ ID NO: 2 - RNA, 31 nt, unidentified
  source 1..31: mol_type = mRNA
agcggccgggcgcgcccgggccagccagggg

SEQ ID NO: 3 - RNA, 672 nt, Synthetic construct
  source 1..672: mol_type = other RNA
gaatttcgccatgatagcggctatgaagtgcatcatggcagcggcagcggcagcggcagcgaagatgtgggcagcaacaaaggcggcagcggcagcggcagcggcagcatggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccg

SEQ ID NO: 4 - RNA, 864 nt, Synthetic construct
  source 1..864: mol_type = other RNA
ctgggcgaaaacgtgaccattgaaagcgtggcggattattttaaacagattggcagcggcagcggcagcggcagcattattaaaaccaacaaaaaaaccggccagggcagcggcagcggcagcggcagccgcgaaaccggcaaactgaaaggcagcggcagcggcagcggcagctttgatggcaaagaatttagcggcggcagcggcagcggcagcggcagccgcgtgaccgaagatgaaaacgatgaaccgattgaaattccgagcgaagatgatggcaccgtgggcagcggcagcggcagcggcagccgctatcgcaacccggtgagccagtgcatgcgcggcgtgggcagcggcagcggcagcggcagccatgcgccggatgcgggctggggcaacggcagcggcagcggcagcggcagcaccaccgaacaggatctgaaagaatattttagcggcagcggcagcggcagcggcagcgaaacccaggtgaaagtgatgagccagcgccatatgattgatggcggcagcggcagcggcagcggcagcccgaacagcaaacagagccaggatgaaccgctgcgcagcggcagcggcagcggcagcggcagcgatatgaccgaagatgaactgcgcgaattttttagcggcagcggcagcggcagcggcagcggcgaagatctgattattaaaggcggcagcggcagcggcagcggcagcgcggaaccgaaacataacagcaaccgccagctggaacgcagcggccgctttggcggcaacccgggcggctttggcagcggcagcggcagcggcagcggctttaacggcggctttggcagcagcatggatagcaaaagcagcggc

SEQ ID NO: 5 - RNA, 564 nt, Synthetic construct
  source 1..564: mol_type = other RNA
atggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccg

SEQ ID NO: 6 - RNA, 1452 nt, Synthetic construct
  source 1..1452: mol_type = other RNA
atggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccgggcagcggcagcggcagcggcagccgcgtgaccgaagatgaaaacgatgaaccgattgaaattccgagcgaagatgatggcaccgtgggcagcggcagcggcagcggcagccgctatcgcaacccggtgagccagtgcatgcgcggcgtgggcagcggcagcggcagcggcagccatgcgccggatgcgggctggggcaacggcagcggcagcggcagcggcagcaccaccgaacaggatctgaaagaatattttagcggcagcggcagcggcagcggcagcgaaacccaggtgaaagtgatgagccagcgccatatgattgatggcggcagcggcagcggcagcggcagcccgaacagcaaacagagccaggatgaaccgctgcgcagcggcagcggcagcggcagcggcagcgatatgaccgaagatgaactgcgcgaattttttagcggcagcggcagcggcagcggcagcggcgaagatctgattattaaaggcggcagcggcagcggcagcggcagcgcggaaccgaaacataacagcaaccgccagctggaacgcagcggccgctttggcggcaacccgggcggctttggcagcggcagcggcagcggcagcggctttaacggcggctttggcagcagcatggatagcaaaagcagcggcggcagcggcagcggcagcggcagcctgggcgaaaacgtgaccattgaaagcgtggcggattattttaaacagattggcagcggcagcggcagcggcagcattattaaaaccaacaaaaaaaccggccagggcagcggcagcggcagcggcagccgcgaaaccggcaaactgaaaggcagcggcagcggcagcggcagctttgatggcaaagaatttagcggc

SEQ ID NO: 7 - RNA, 912 nt, Synthetic construct
  source 1..912: mol_type = other RNA
aaaggcctgagcaaaggcagcggcagcggcagcggcagcgaaaaaaccaaacagggcgtggcggaagcggcgggcaaaaccaaagaaggcgtgggcagcggcagcggcagcggcagcgtgggcagcaaaaccaaagaaggcgtggtgggcagcggcagcggcagcggcagcgcggaaaaaaccaaagaacaggtgaccaacgtgggcggcgcggtggtgaccggcggcagcggcagcggcagcggcagcaccgcggtggcgcagaaaaccgtggaaggcagcggcagcggcagcggcagctttgtgaaaaaagatcagctgggcggcagcggcagcggcagcggcagcatggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccg

SEQ ID NO: 8 - RNA, 324 nt, Synthetic construct
  source 1..324: mol_type = other RNA
aaaggcctgagcaaaggcagcggcagcggcagcggcagcgaaaaaaccaaacagggcgtggcggaagcggcgggcaaaaccaaagaaggcgtgggcagcggcagcggcagcggcagcgtgggcagcaaaaccaaagaaggcgtggtgggcagcggcagcggcagcggcagcgcggaaaaaaccaaagaacaggtgaccaacgtgggcggcgcggtggtgaccggcggcagcggcagcggcagcggcagcaccgcggtggcgcagaaaaccgtggaaggcagcggcagcggcagcggcagctttgtgaaaaaagatcagctgggc

SEQ ID NO: 9 - RNA, 912 nt, Synthetic construct; identical to SEQ ID NO: 7
  source 1..912: mol_type = other RNA

SEQ ID NO: 10 - RNA, 564 nt, Synthetic construct; identical to SEQ ID NO: 5
  source 1..564: mol_type = other RNA

SEQ ID NO: 11 - protein, 12 aa, Synthetic construct
EFRHDSGYEVHH

SEQ ID NO: 12 - protein, 8 aa, Synthetic construct
EDVGSNKG

SEQ ID NO: 13 - protein, 9 aa, Synthetic construct
MAEPRQEFE

SEQ ID NO: 14 - protein, 9 aa, synthetic construct
NAKAKTDHG

SEQ ID NO: 15 - protein, 10 aa, Synthetic construct
LREPGPPGLS

SEQ ID NO: 16 - protein, 11 aa, Synthetic construct
THVPGGGNKKI

SEQ ID NO: 17 - protein, 12 aa, Synthetic construct
HHKPGGGQVEVK

SEQ ID NO: 18 - protein, 15 aa, Synthetic construct
TPNVQKEQAHSEEHL

SEQ ID NO: 19 - protein, 18 aa, Synthetic construct
SKIGSTENLKHQPGGGKV

SEQ ID NO: 20 - protein, 18 aa, Synthetic construct
SKCGSKDNIKHVPGGGSV

SEQ ID NO: 21 - protein, 22 aa, Synthetic construct
TEIPASEPDGPSVGRAKGQDAP

SEQ ID NO: 22 - protein, 5 aa, Synthetic construct
KGLSK

SEQ ID NO: 23 - protein, 18 aa, Synthetic construct
EKTKQGVAEAAGKTKEGV

SEQ ID NO: 24 - protein, 10 aa, Synthetic construct
VGSKTKEGVV

SEQ ID NO: 25 - protein, 18 aa, Synthetic construct
AEKTKEQVTNVGGAVVTG

SEQ ID NO: 26 - protein, 17 aa, Synthetic construct
TAVAQKTVEFVKKDQLG

SEQ ID NO: 27 - protein, 21 aa, Synthetic construct
RVTEDENDEPIEIPSEDDGTV

SEQ ID NO: 28 - protein, 13 aa, Synthetic construct
RYRNPVSQCMRGV

SEQ ID NO: 29 - protein, 9 aa, Synthetic construct
HAPDAGWGN

SEQ ID NO: 30 - protein, 11 aa, Synthetic construct
TTEQDLKEYFS

SEQ ID NO: 31 - protein, 15 aa, Synthetic construct
ETQVKVMSQRHMIDG

SEQ ID NO: 32 - protein, 13 aa, Synthetic construct
PNSKQSQDEPLRS

SEQ ID NO: 33 - protein, 12 aa, Synthetic construct
DMTEDELREFFS

SEQ ID NO: 34 - protein, 8 aa, Synthetic construct
GEDLIIKG

SEQ ID NO: 35 - protein, 24 aa, Synthetic construct
AEPKHNSNRQLERSGRFGGNPGGF

SEQ ID NO: 36 - protein, 16 aa, Synthetic construct
GFNGGFGSSMDSKSSG

SEQ ID NO: 37 - protein, 17 aa, Synthetic construct
LGENVTIESVADYFKQI

SEQ ID NO: 38 - protein, 10 aa, Synthetic construct
IIKTNKKTGQ

SEQ ID NO: 39 - protein, 7 aa, Synthetic construct
RETGKLK

SEQ ID NO: 40 - protein, 8 aa, Synthetic construct
FDGKEFSG

SEQ ID NO: 41 - RNA, 216 nt, unidentified
  source 1..216: mol_type = mRNA
gcagcgcccccccgccgggaccccgagccccccgacccgggcccaggagccccaccccaccgccccaccaccacccgcagccagacacccccaagcacgcagcaagcagccaaaacgcagccagccacacccccacgggaaacagcaggaaaccagcaaaaacgaaagaacaagcaacaaccccagggggcaacggccagccacacccggagcagc

SEQ ID NO: 42 - RNA, 107 nt, Synthetic construct
  source 1..107: mol_type = other RNA
aaaaaaaaaaaaaaaaaaaaaaaaaaaaaagcaagacaaaaaaaaaaaaaaaaaaaaaaaaaaaaaaaaaaaaaaaaaaaaaaaaaaaaaaaaaaaaaaaaaaaaaa